(19) United States
(12) Patent Application Publication
(10) Pub. No.: US 2026/0144663 A1
(43) Pub. Date: May 28, 2026

(54) Adjustable Orthosis

(71) Applicant: Outcome-Based Technologies LLC, Cheyenne, WY (US)

(72) Inventors: Donald P. LoGuercio, Scottsdale, AZ (US); Arni Thor, Coronado, CA (US)

(73) Assignee: Outcome-Based Technologies LLC, Cheyenne, WY (US)

(21) Appl. No.: 19/403,648

(22) Filed: Nov. 28, 2025

Related U.S. Application Data
(60) Provisional application No. 63/726,205, filed on Nov. 27, 2024.
(60) Provisional application No. 63/823,602, filed on Jun. 13, 2025.

(51) Int. Cl. A61F 5/055

(52) U.S. Cl. CPC A61F 5/055

(57) ABSTRACT

An adjustable orthosis includes a lower collar configured to rest on a patient torso and an upper collar configured to support a patient head. An arm extends between the lower collar and the upper collar and supports the upper collar at an elevated position with respect to the lower collar. The arm enables rotational, pitching, and vertical movement of the upper collar with respect to the lower collar through ranges of movement in three planes.

### CROSS-REFERENCE TO RELATED APPLICATIONS

This application claims the benefit of U.S. Provisional Application No. 63/726,205, filed Nov. 27, 2024, and of U.S. Provisional Application No. 63/823,602, filed Jun. 13, 2025, both of which are hereby incorporated by reference in their entireties.

### FIELD

The present specification relates generally to medicine, health, and physical and rehabilitative therapy, and more particularly to orthopedic devices.

### BACKGROUND

Orthoses, such as cervical collars, knee braces, arm splints, hip orthotics, and others, are highly-specialized pieces of medical equipment used to provide post-operative support and rehabilitation. Conventionally, orthoses act generally to stabilize and restrict movement of a body part or joint in order to promote healing.

Conventional orthoses have a number of limitations, however. The design of an orthosis is time-consuming, expensive, and skill-intensive. Using a cervical collar as an example, generally, a doctor must custom-fit a collar to a patient. This requires measuring the patient very carefully, usually with a three-dimensional scan of the patient's upper chest, neck, and lower jaw or head. Sometimes a specialist will perform this scan. The scan will then be sent to a mill, where a machine cuts down a block of foam into the size and shape of the patient's body. The milling machine is a CNC machine or similar machine, and it uses a rotating bit to shave off layers of foam. Often, this means that the foam mold will have a corrugated outer surface. In other words, there will be waves, ripples, or small channels covering the entire outer surface of the finished mold. A human technician then has to shave or sand down the crests between the channels, so that the mold is rendered smooth.

This foam mold is then sent to a specialized factory, which constructs a custom cervical collar for the patient based on the foam mold. The factory will build the collar specifically and uniquely for the foam mold. Therefore, if in the process of shaving or sanding the foam mold down to reduce the crests, the foam mold is too small, the resulting cervical collar may also be too small. Because of this, creating a custom cervical collar can be difficult and expensive.

Once created and fitted, most cervical collars fully restrain movement. There are a number of associated problems with this. Moreover, once the collar is removed at the conclusion of therapy or treatment, patients transition abruptly to no collar at all, leaving most feeling unprotected and anxious.

Problems such as these are not isolated to cervical collars; they exist for all manner of orthopedic devices. There is a need for a better process and solution.

### SUMMARY

In an embodiment, an adjustable orthosis includes a lower collar configured to rest on a patient torso and an upper collar configured to support a patient head. An arm extends between the lower collar and the upper collar, the arm supporting the upper collar at an elevated position with respect to the lower collar, and the arm enables rotational movement of the upper collar with respect to the lower collar through a rotational range of movement in a transverse plane.

In embodiments, a track is formed in the lower collar and has opposed first and second ends, wherein the arm is carried in the track for movement through the rotational range of movement between the first and second ends of the track. First and second stops are applicable to the track such that, when the first and second stops are applied to the track between the first and second ends, the first and second stops limit the rotational range of movement to between the first and second stops. The arm further enables pitching movement of the upper collar with respect to the lower collar through a range of movement in a sagittal plane. The arm includes a post coupled to the lower collar, a strut coupled to the upper collar, a spindle defining an axis of rotation for the pitching movement wherein the spindle is fixed with respect to the strut, an inner plate mounted for rotation on the spindle, and an outer plate mounted for rotation on the spindle, and the post is mounted for rotation on the spindle with respect to the strut. The inner plate has a first set of teeth and a block outboard of the first set of teeth, the outer plate has a second set of teeth engaged with the first set of teeth and a second block outboard of the second set of teeth, and the post has a third set of teeth engaged with the second set of teeth. The spindle is carried on a base plate and the base plate includes a third block, during pitching movement the base plate rotates on the strut with respect to the post and the third block moves between the first and second blocks, wherein the first and second blocks confront the third block to limit rotational movement of the base plate and the strut with respect to the post. The arm further enables vertical movement of the upper collar with respect to the lower collar through a vertical range of movement in a frontal plane. The strut includes a channel and the base plate includes a rail fit into the channel, wherein, during vertical movement of the upper collar with respect to the lower collar, the rail of the base plate slides within the channel of the strut and the post is coupled to the base plate by the inner and outer plates such that movement of the base plate with respect to the strut imparts movement of the post with respect to the strut. Stops are carried by the base plate which confront the strut to limit vertical movement of the upper collar with respect to the lower collar to within a defined range.

In an embodiment, an adjustable orthosis includes a lower collar configured to rest on a patient torso and an upper collar configured to support a patient head. An arm extends between the lower collar and the upper collar, the arm supporting the upper collar at an elevated position with respect to the lower collar, and the arm enables pitching movement of the upper collar with respect to the lower collar through a pitching range of movement in a sagittal plane.

In embodiments, the arm includes a post coupled to the lower collar, a strut coupled to the upper collar, and a spindle carried on the strut, the spindle defining an axis of rotation for the pitching movement, and the post is mounted for rotation on the spindle with respect to the strut. Inner and outer plates are positioned between the post and the strut, the inner plate has a first set of teeth and a block outboard of the first set of teeth, the outer plate has a second set of teeth engaged with the first set of teeth and a second block outboard of the second set of teeth, and the post has a third set of teeth engaged with the second set of teeth. A base plate is included and has a third block, wherein, during pitching movement, the base plate rotates on the strut with respect to the post and the third block moves between the first and second blocks, wherein the first and second blocks confront the third block to limit rotational movement of the base plate and the strut with respect to the post. Bands extend between the post and the strut and impart a bias therebetween. A pitch assembly is provided in the arm and first and second stops are applicable to the arm such that, when the stops are applied to the pitch assembly, the stops limit the pitching range of movement to between the first and second stops. The arm further enables vertical movement of the upper collar with respect to the lower collar through a vertical range of movement in a frontal plane. The strut includes a channel and the base plate includes a rail fit into the channel, wherein, during vertical movement of the upper collar with respect to the lower collar, the rail of the base plate slides within the channel of the strut and the post is coupled to the base plate by the inner and outer plates such that movement of the base plate with respect to the strut imparts movement of the post with respect to the strut.

In an embodiment, an adjustable orthosis includes a lower collar configured to rest on a patient torso, an upper collar configured to support a patient head, and an arm extending between the lower and upper collars, the arm supporting the upper collar at an elevated position with respect to the lower collar. The arm supports the upper collar at an elevated position with respect to the lower collar and enables a range of movement of the upper collar with respect to the lower collar in at least one of three planes of movement. The arm includes stops to limit the range of movement of the upper collar within a defined range.

The above provides the reader with a brief summary of some embodiments described below. Simplifications and omissions are made, and the summary is not intended to limit or define in any way the disclosure. Rather, this brief summary merely introduces the reader to some aspects of some embodiments in preparation for the detailed description that follows.

### BRIEF DESCRIPTION OF THE DRAWINGS

### Referring to the Drawings:

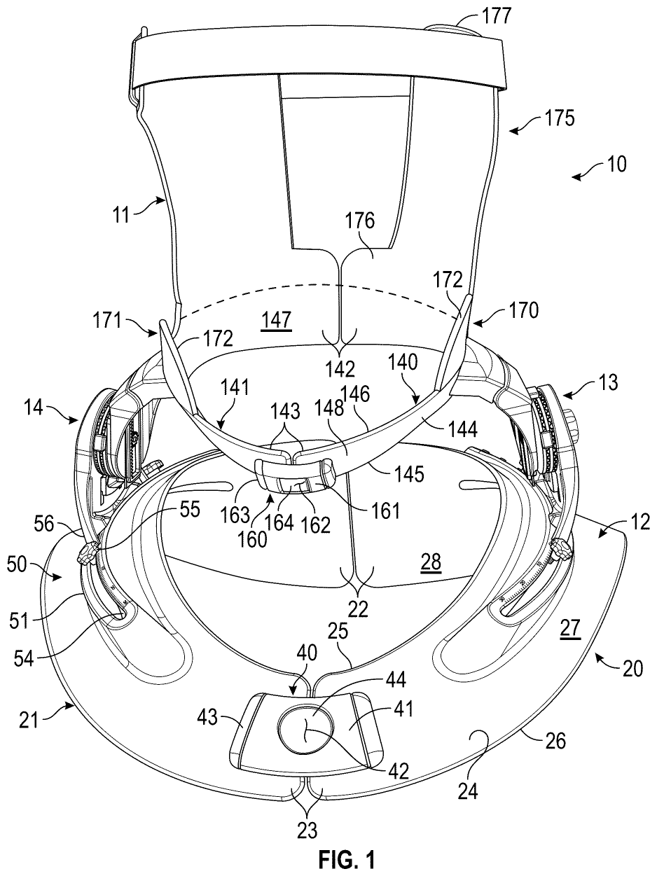

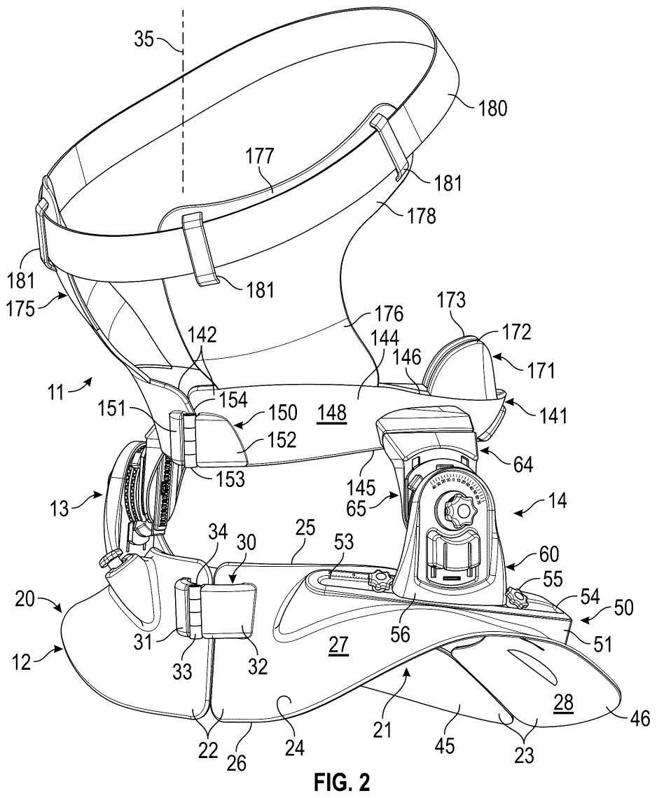

FIGS. 1 and 2 are front and rear perspective views of an embodiment of an adjustable orthosis having upper and lower collars;

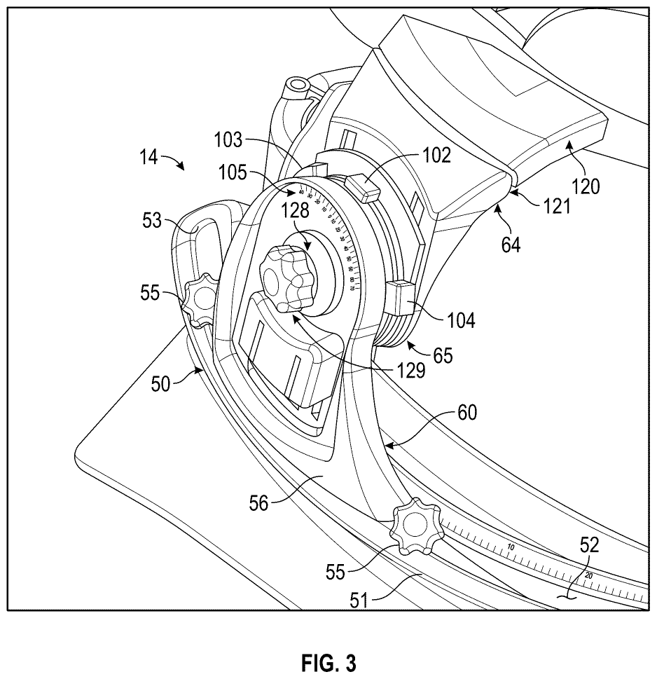

FIG. 3 is an enlarged view of the orthosis of FIG. 1, illustrating an arm between the upper and lower collars;

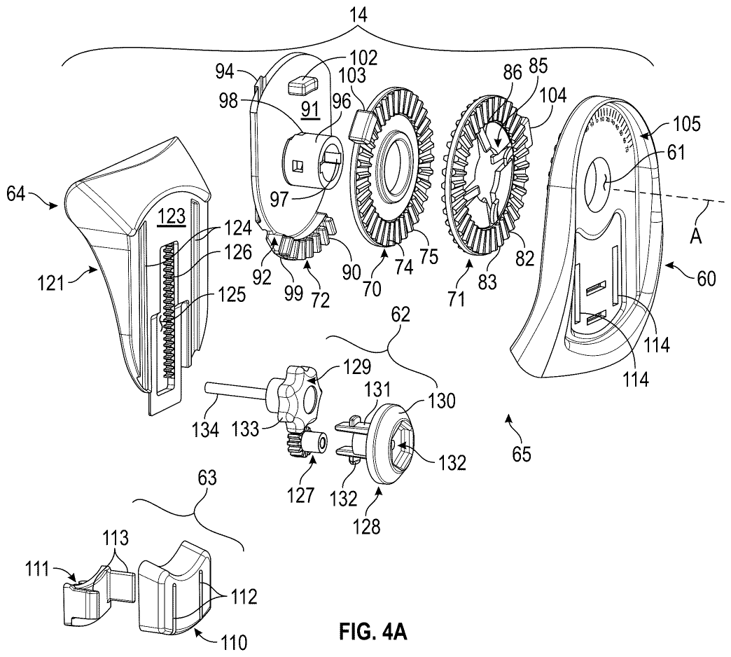

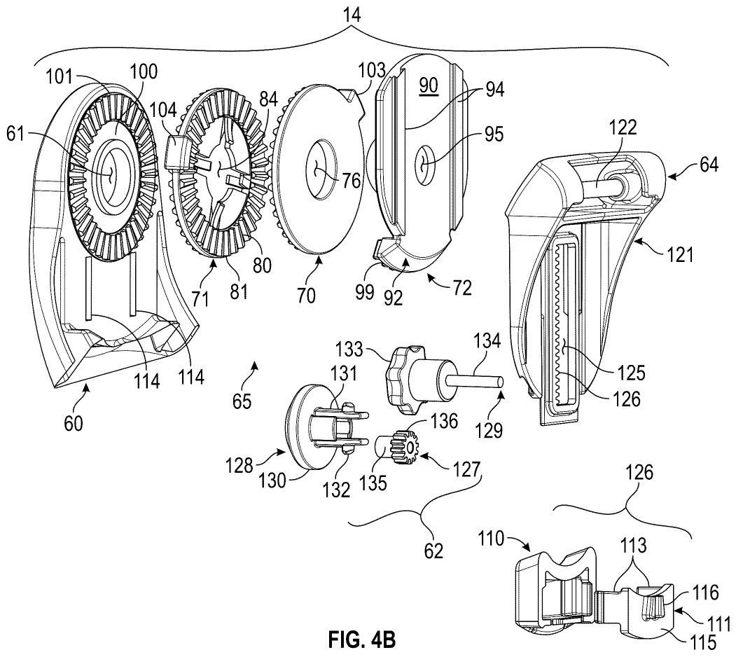

FIGS. 4A and 4B are left and right exploded views of the arm of FIG. 1;

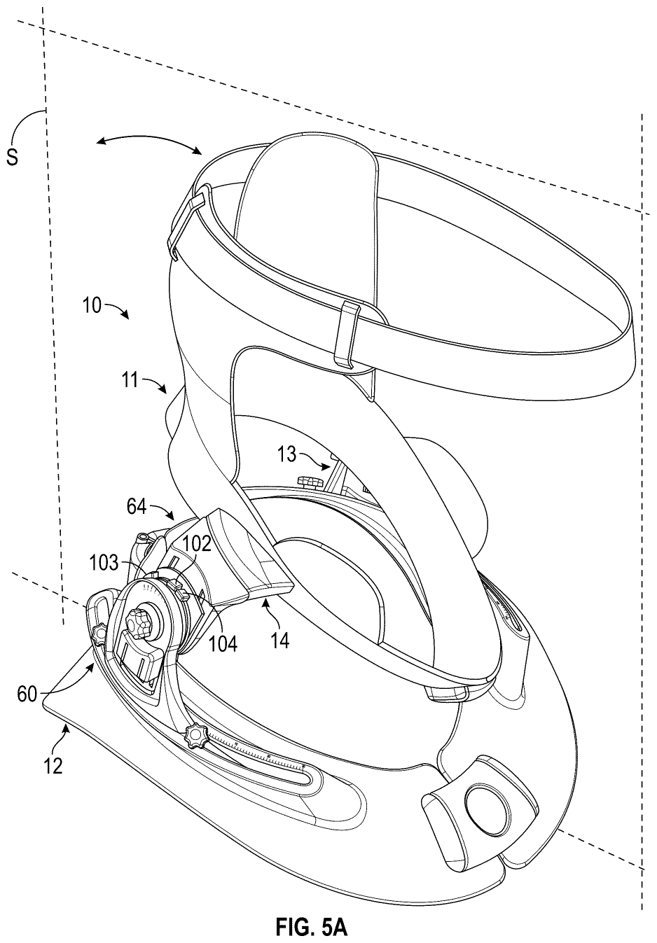

FIGS. 5A and 5B illustrate the orthosis of FIG. 1 in pitching movement;

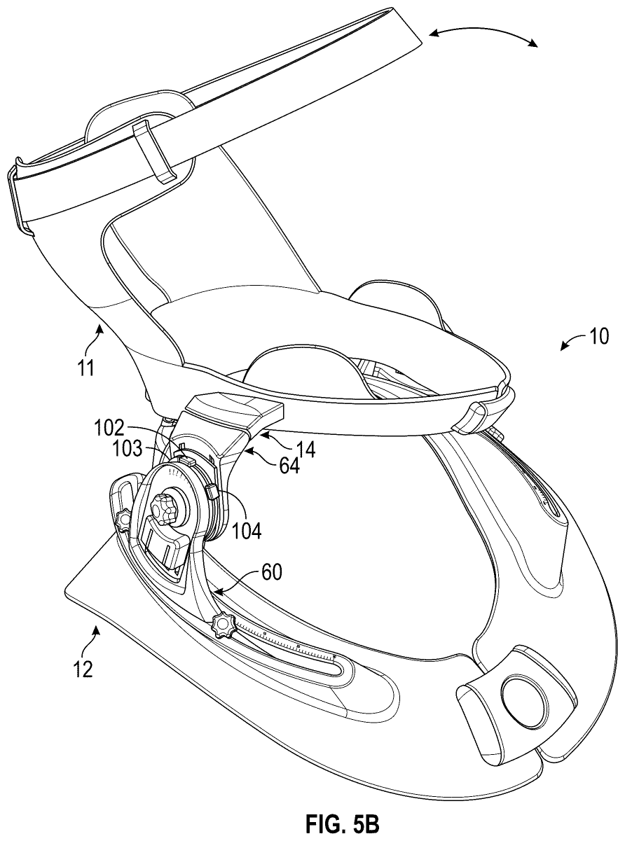

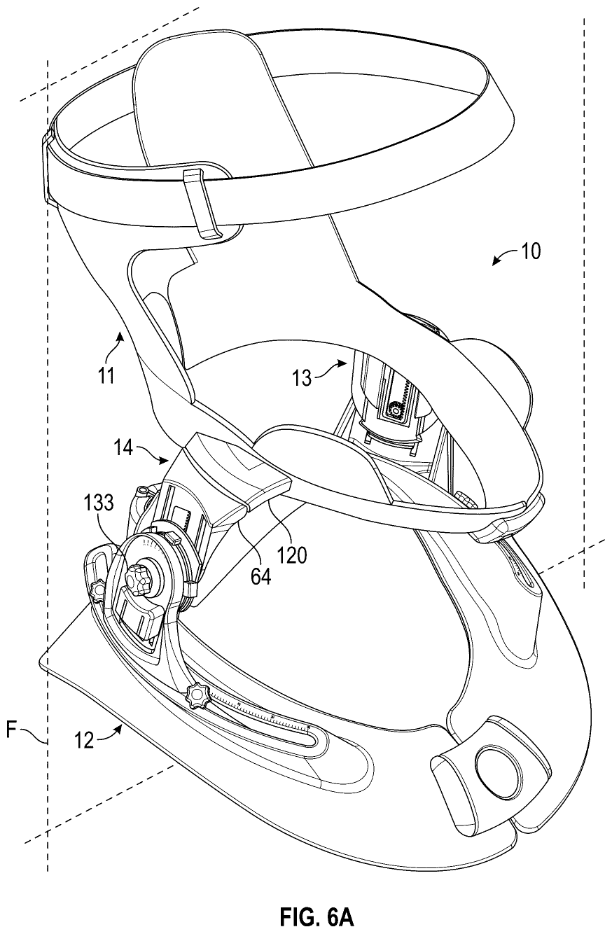

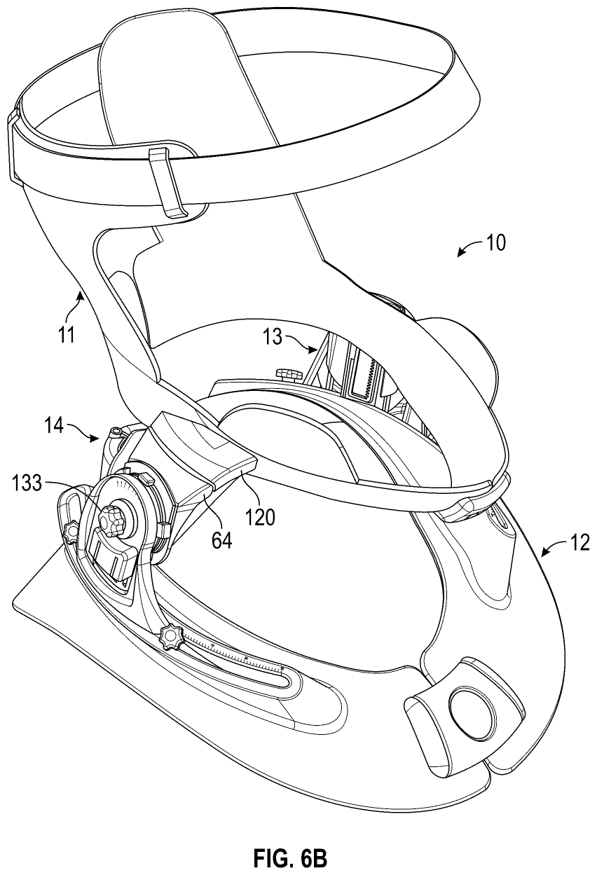

FIGS. 6A and 6B illustrate the orthosis of FIG. 1 in vertical movement;

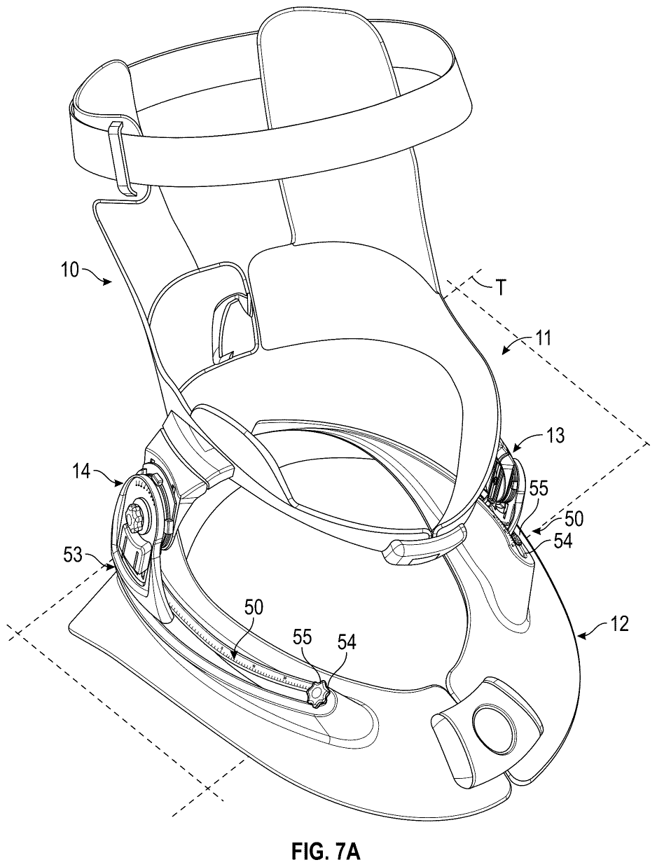

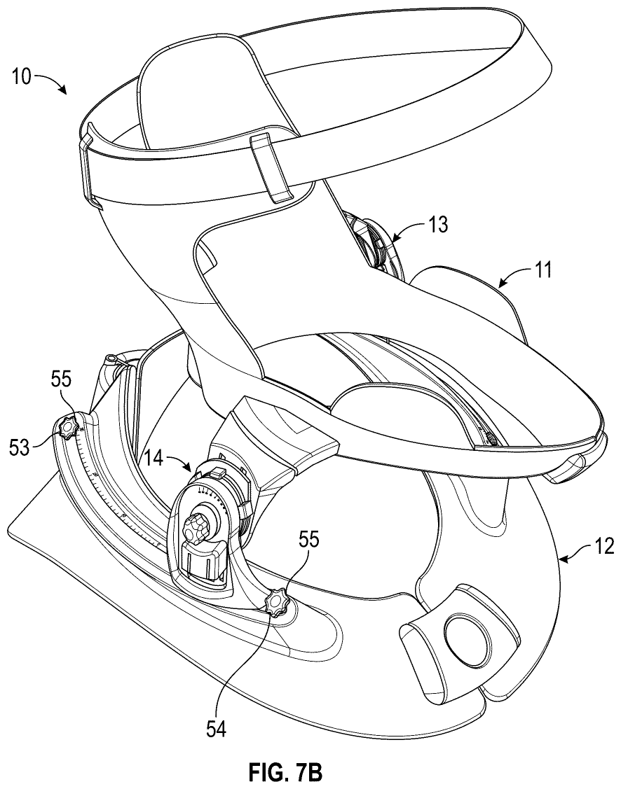

FIGS. 7A and 7B illustrate the orthosis of FIG. 1 in rotational movement;

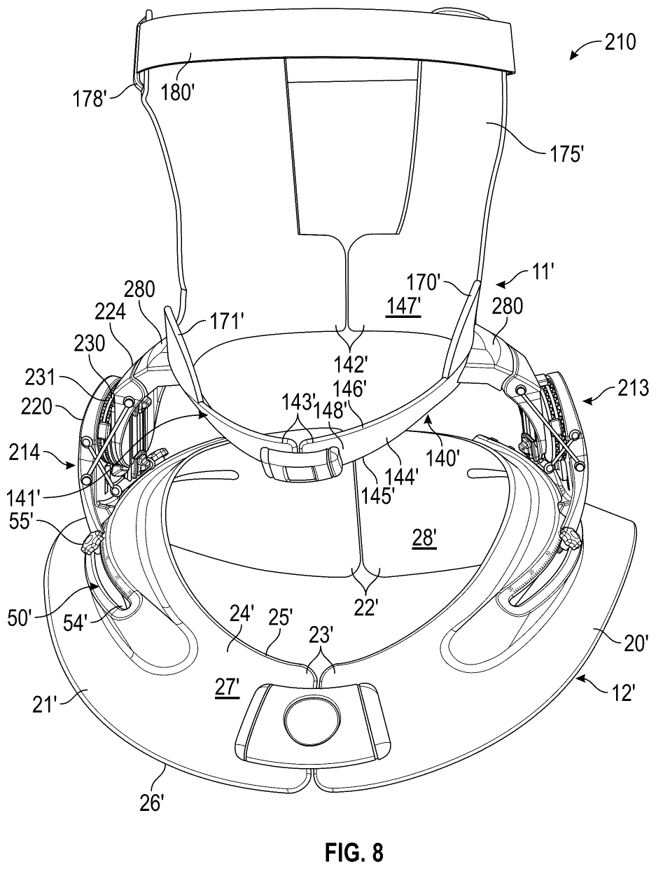

FIG. 8 is a front perspective view of an embodiment of an adjustable cervical collar orthosis having upper and lower collars;

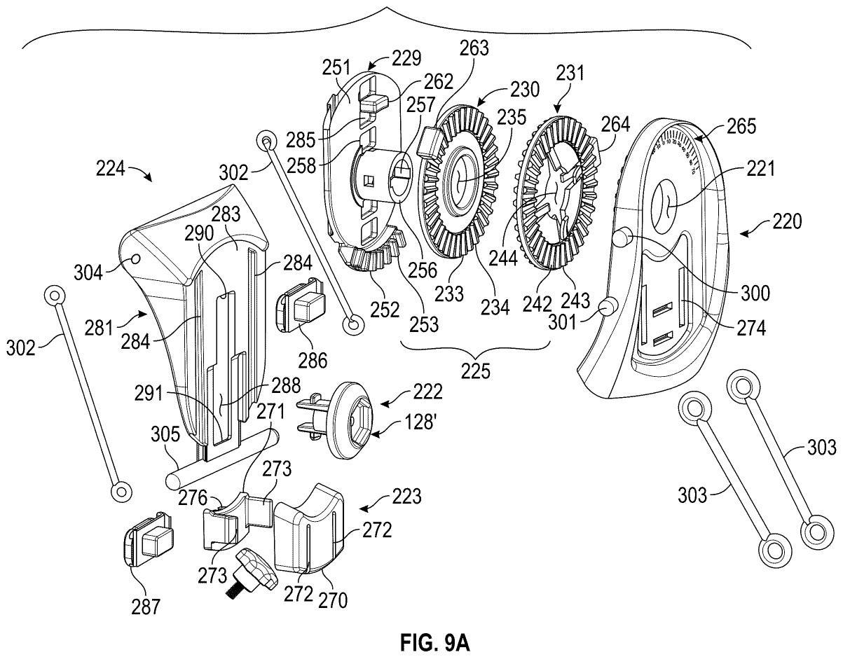

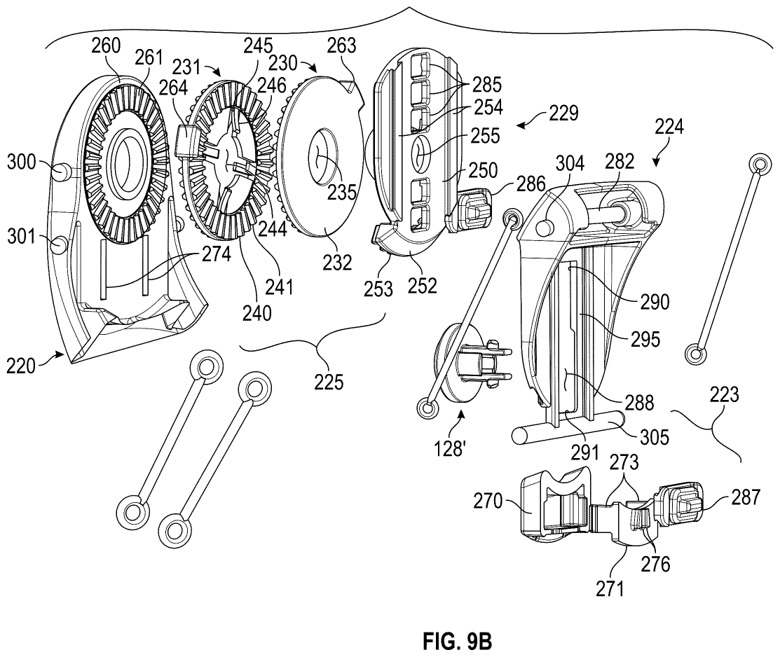

FIGS. 9A and 9B are left and right exploded views of the an of the orthosis of FIG. 8;

FIGS. 10A and 10B illustrate the orthosis of FIG. 8 in pitching movement;

FIGS. 11A and 11B illustrate the orthosis of FIG. 8 in vertical movement;

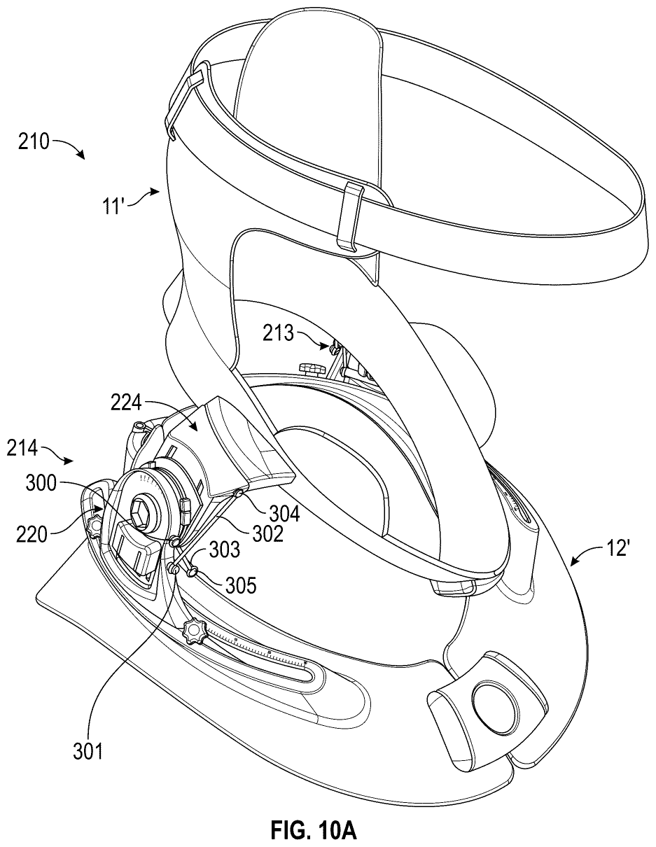

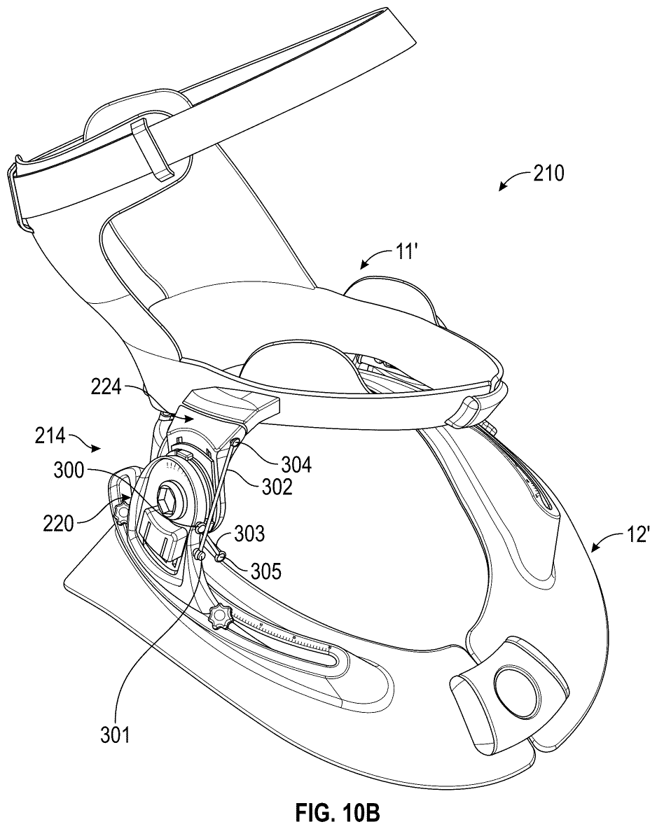

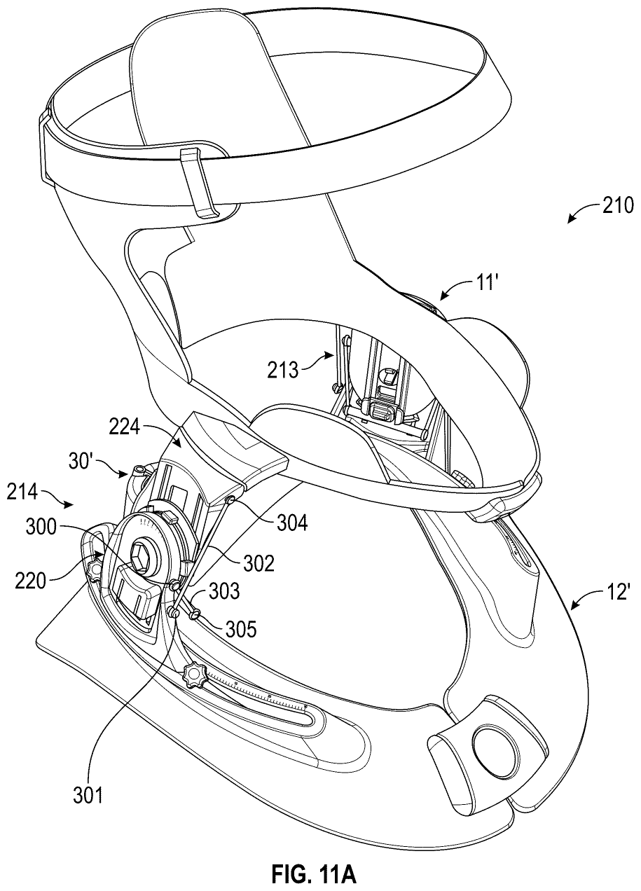

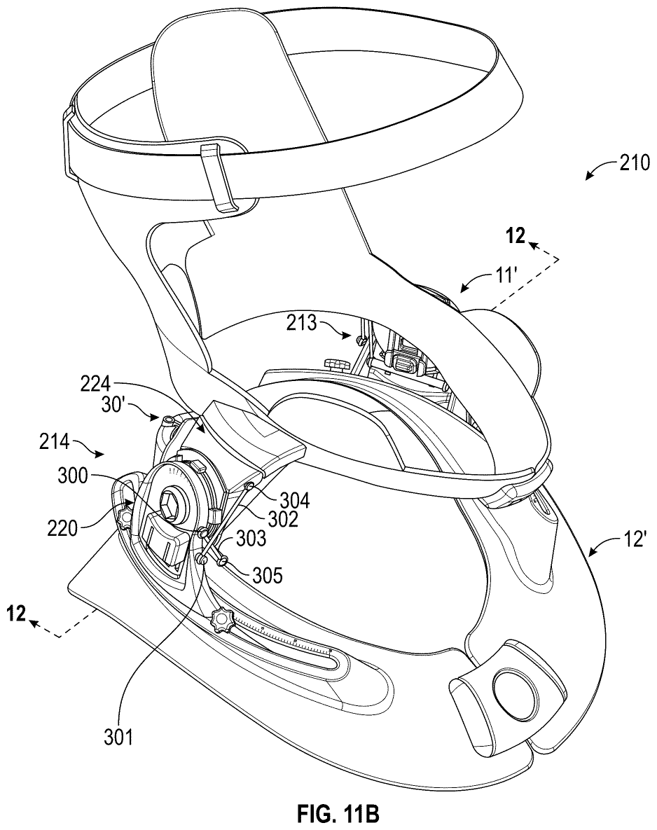

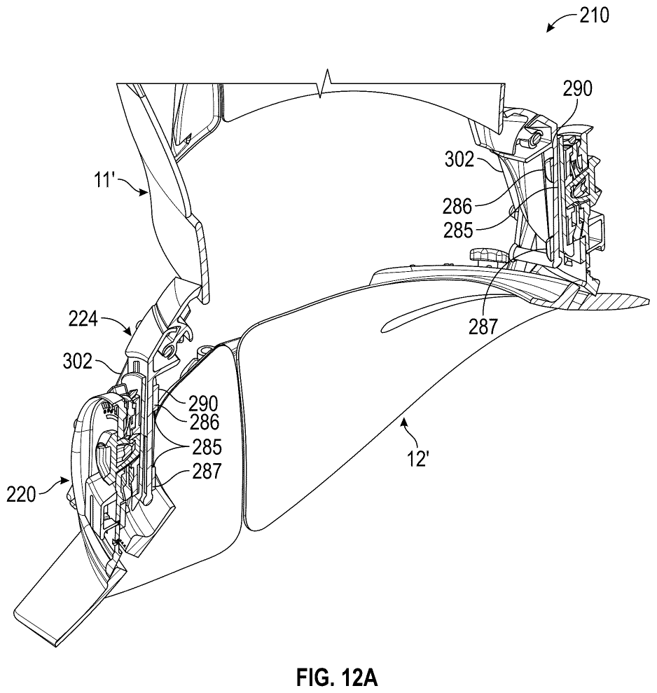

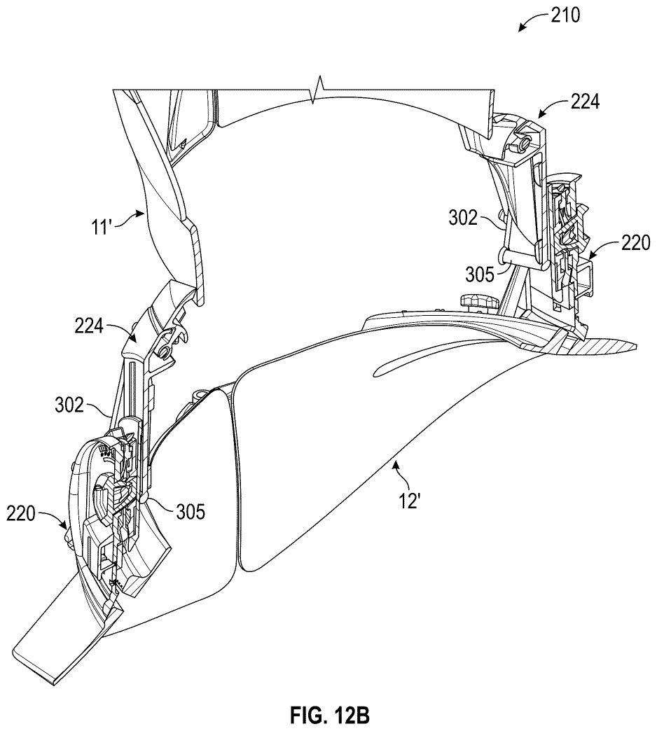

FIGS. 12A and 12B are section views of the orthosis of FIG. 8, taken along the line 12-12, showing vertical movement thereof;

FIG. 13 is a perspective view of positive mold of a patient's neck area; and

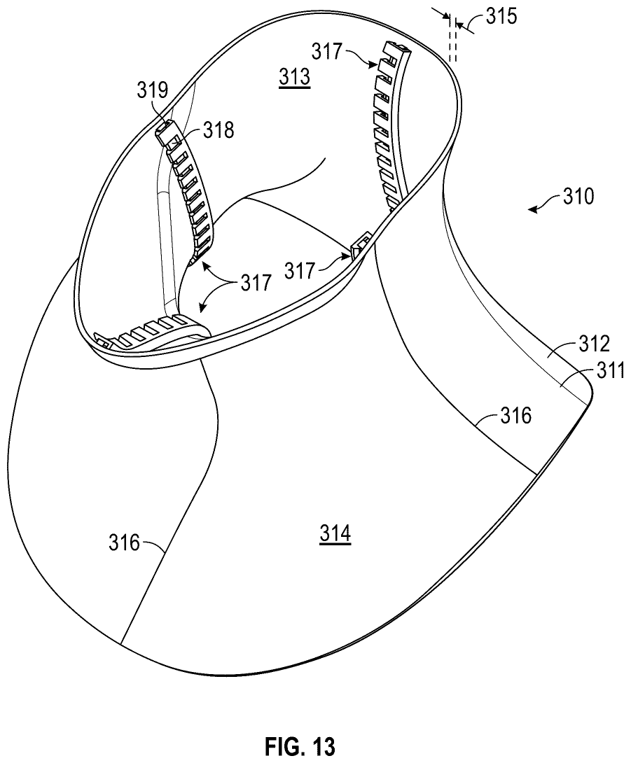

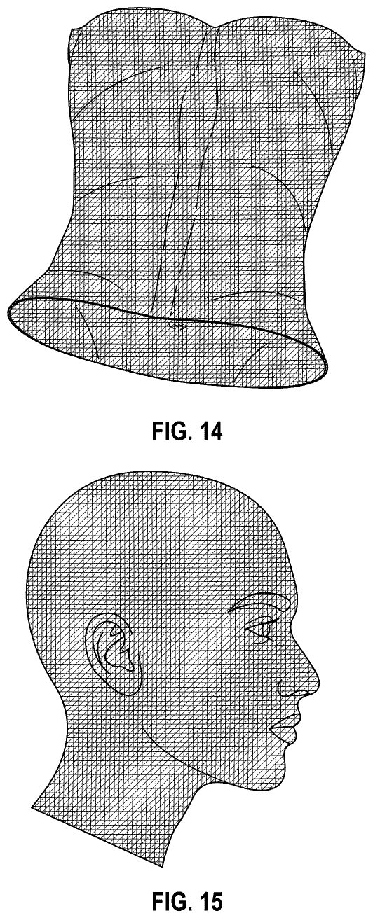

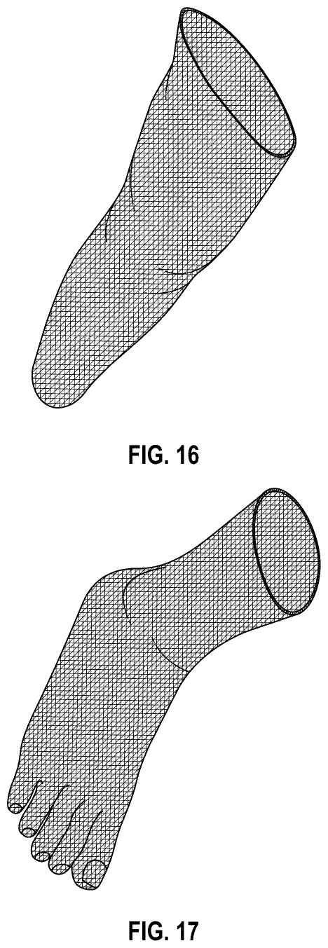

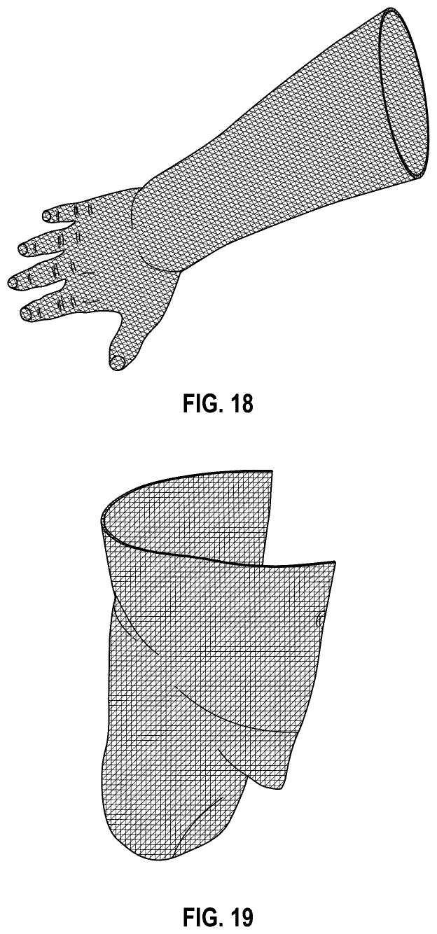

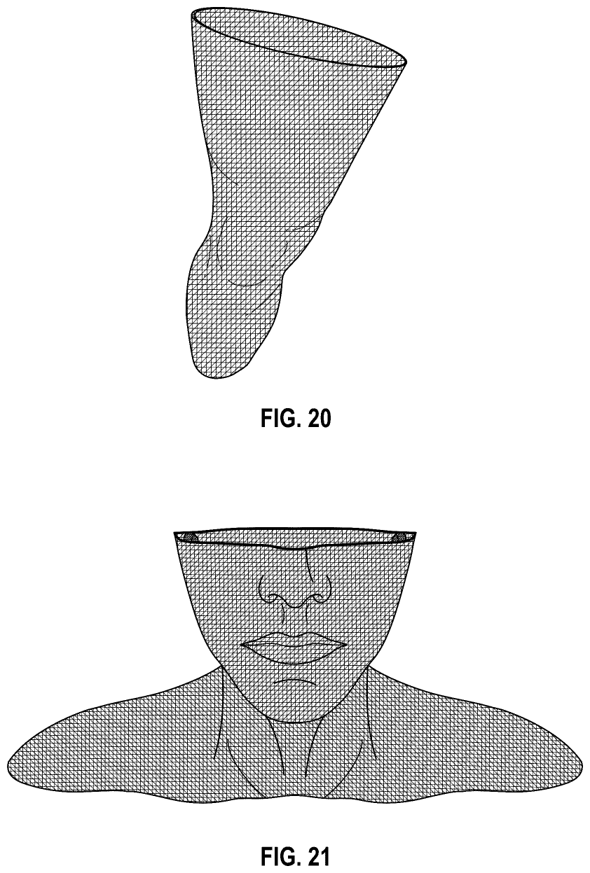

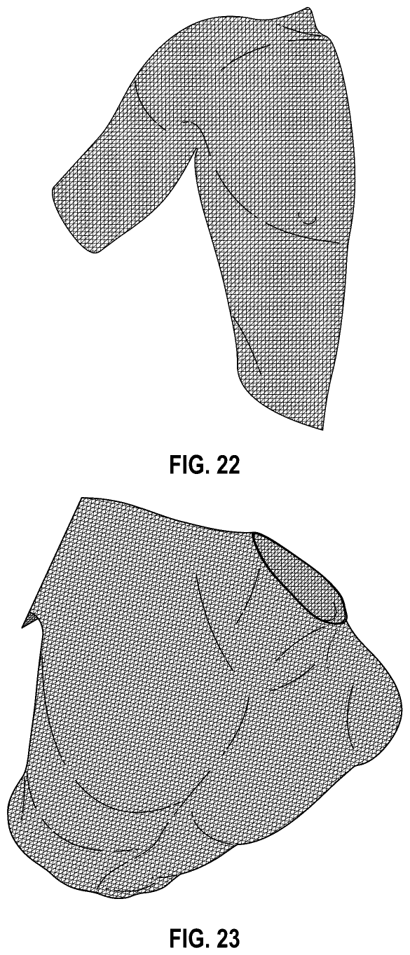

FIGS. 14-23 are perspective views of positive molds of other body parts and sections of a patient.

### DETAILED DESCRIPTION

Reference now is made to the drawings, in which the same reference characters are used throughout the different figures to designate the same elements. Briefly, the embodiments presented herein are preferred exemplary embodiments and are not intended to limit the scope, applicability, or configuration of all possible embodiments, but rather to provide an enabling description for all possible embodiments within the scope and spirit of the specification. Description of these preferred embodiments is generally made with the use of verbs such as “is” and “are” rather than “may,” “could,” “includes,” “comprises,” and the like, because the description is made with reference to the drawings presented. One having ordinary skill in the art will understand that changes may be made in the structure, arrangement, number, and function of elements and features without departing from the scope and spirit of the specification. Further, the description may omit certain information which is readily known to one having ordinary skill in the art to prevent crowding the description with detail which is not necessary for enablement. Indeed, the diction used herein is meant to be readable and informational rather than to delineate and limit the specification; therefore, the scope and spirit of the specification should not be limited by the following description and its language choices.

FIGS. 1-7B illustrate an exemplary embodiment of an adjustable cervical collar orthosis 10 (hereinafter, “orthosis 10”). The orthosis 10 includes a cranial support ring or upper collar 11, a lower base or lower collar 12, and left and right armatures or support arms 13 and 14, respectively, extending between the upper and lower collars 11 and 12 to support the upper collar 11 at an elevated position with respect to the lower collar 12. The upper collar 11 is held in vertically spaced relation with respect to the lower collar 12 by the arms 13 and 14. Those arms 13 and 14 set the height of the upper collar 11 above the lower collar 12, establish the pitch (the flexion and extension) of the upper collar 11 with respect to the lower collar 12, determine the roll of the upper collar 11 with respect to the lower collar 12, and control the rotation of the upper collar 11 with respect to the lower collar 12. The upper and lower collars 11 and 12 are entirely customizable. The lower collar 12 is formed to match the features and curvature of a patient's upper torso and neck area so that it may rest thereon, and the upper collar 11 is formed to match exactly the features and curvature of a patient's neck, chin, and head. Moreover, the placement or location of the left and right arms 13 and 14 along the upper and lower collars 11 and 12 can be changed to fit the patient's anatomy, functional requirement, and specific needs.

The lower collar 12 is a wide base for the orthosis 10. It distributes the weight and pressure of the orthosis 10 across the sternum, the upper back, the clavicles, trapezius muscles, and other upper portions of the torso. This helps prevent hot spots and sores on the body and also helps prevent degradation of the orthosis 10 itself.

The lower collar 12 is preferably formed from two shells. In the embodiment shown in these drawings, those shells include a left shell 20 and a right shell 21. In other embodiments, the shells are posterior and anterior shells, and in other embodiments, there are other shells of other numbers and configurations. Here, the two shells 20 and 21 are identical and mirror opposites. As such, reference is made herein to either shell without distinction, and the same reference characters are used to identify the same structural elements and features in the shells 20 and 21. Where the description refers to one of the shells 20 or 21, the reader will understand that the description applies equally to the other of the shells 21 or 20 unless specifically noted. The shells 20 and 21 are shown in FIGS. 1 and 2.

The shell 21 has a rear 22, a front 23, and a body 24 extending therebetween. The body 24 is thin and wide. It has a width extending between an inner edge 25 and an outer edge 26, and it has an upper surface 27 and an opposed lower surface 28. The width between the inner and outer edges 25 and 26 is much greater than the thickness between the upper and lower surfaces 27 and 28. Very roughly, and without limitation, the width of the body 24 may preferably be between one and three inches, though in other embodiments it may be more or less. Because the orthosis 10 is custom constructed for a patient, some patients may need or want a narrower or wider lower collar 12, and some health workers may order lower collars 12 of different sizes for specific needs or functions. Regardless, generally, the width of the lower collar 12 is much greater than its thickness.

The two rears 22 of the left and right shells 20 and 21 abut each other and are coupled to each other at a hinge 30. FIG. 3 shows the hinge 30. The hinge 30 includes a left leaf 31 on the left shell 20 and a right leaf 32 on the right shell 21. The leaves 31 and 32 engage with each other to form a knuckle 33 in which a pin 34 sits and defines an axis 35 of pivotal movement of the two shells 20 and 21 with respect to each other. The axis 35 is shown in FIG. 2 as a straight, albeit broken, line.

At the fronts 23, the left and right shells 20 and 21 also couple to each other. Referring to FIG. 1 again, the left and right shells 20 and 21 connect at their fronts 23 with a clasp 40. In the embodiment shown herein, the clasp 40 includes a low dome 41 with a central circular hole 42. The dome 41 is fixed to the left shell 20 but extends partially beyond the front 23 of the shell 20, such that the hole 42 is disposed partially over the shell 20 and partially off the shell 20. The right shell 21 carries a tongue 43 which projects off the front 23 of the right shell 21 toward the left shell 20. The tongue 43 terminates in an enlarged circular raised head 44 which is sized and shaped to snap into the hole 42 of the left shell 20. When the fronts 23 of the shells 20 and 21 are brought together, the tongue 43 moves into the dome 41 and the head 44 snaps into the hole 42, thereby securing the clasp 40. The clasp 40 will not come unbuckled unless a person depresses the head 44 from the hole 42, allowing the tongue 43 to be removed from the dome 41 and the two shells 20 and 21 to be separated from each other. In other embodiments, instead of a clasp 40, the orthosis 10 closes at the lateral interface regions such as overlapping tongues and grooves 186 and one or more fasteners 187 (e.g., magnetic buckles, quick release latches, screws, or cam locks).

The lower surfaces 28 of the left and right shells 20 and 21 carry padding elements 45 and 46. The padding elements 45 and 46 are not quite identical and mirror opposites. The padding element 45 is a thin pad, applied across the entire lower surface 28 of the left shell 20. The padding element 45, however, also preferably extends beyond the rear 22 and front 23 of the left shell 20, such that, when the shells 20 and 21 are coupled and closed, the padding element 45 extends along portions of the lower surface 28 of the right shell 21 inboard of the rear 22 and front 23. In this way, the padding element 45 covers the locations at which the left and right shells 20 and 21 meet, thereby minimizing contact with rough edges which might wear against a patient's skin. While the padding element 45 is adhered or otherwise applied to the lower surface 28 of the left shell 20, it is preferably not applied to the lower surface 28 of the right shell 21.

Conversely, the padding element 46 is preferably applied across less than the entire lower surface 28 of the right shell 21. It is smaller than the padding element 45 and has ends which are inboard of the rear 22 and front 23 of the right shell 21. The entire upper face of the padding element 46 is adhered to or otherwise applied to the lower surface 28 of the right shell 21.

The left and right arms 13 and 14 are coupled to the lower collar 12 for movement. The left and right arms 13 and 14 are identical and mirror opposites. As such, reference is made herein to either or both arms without distinction, and the same reference characters are used to identify the same structural elements and features in the arms 13 and 14. Where the description refers to one of the arms 13 or 14, the reader will understand that the description applies equally to the other of the arms 14 or 13 unless specifically noted. The reader should further understand that, while the drawings show two arms 13 and 14, some embodiments or the orthosis 10 incorporate only a single arm—either arm 13 or arm 14—rather than two arms. For the purpose of disclosure, however, this description refers to an embodiment with two arms. The arms 13 and 14 are shown in FIGS. 1-3 and in exploded view in FIGS. 4A and 4B.

The right shell 21 includes a track 50 formed on the outer surface 28. The track 50 is arcuate and extends a majority of the length of the right shell 21 between the rear 22 and the front 23. The track 50 projects upward. It has an upstanding sidewall 51 that terminates in a top formed with an elongate slot 52 extending between a rear end 53 and a front end 54. A bottom 56 of the right arm 14 is carried in the track for sliding, reciprocating movement along the track 50 between the rear and front ends 53 and 54. Threaded set screws 55 threadably engage with the slot 52 to tighten and loosen in the slot 52. The screws 55 can be loosened, slid along the track 50 to a desired position, and then tightened and secured in the desired position. In this way, the screws 55 are stops which limit a range of movement of the right arm 14 in the track 50 to between the screws 55 rather than between the full extent of the track 50 itself.

The right arm 14 includes a base post, or post 60, with a tongue carried in the track 50. The tongue is enlarged such that it cannot be removed from the slot 52. The right arm 14 is thus mounted for reciprocal movement in the track 50.

The post 60 projects upwardly from the lower collar 12 at approximately two-thirds the distance from the front 23 to the rear 22 of the lower collar 12. The post 60 is slender, having opposed major faces directed inward toward the patient and outward away from the patient. The post 60 has a peripheral edge that arcuately bends upward from and then downward to the lower collar 12, roughly along a line extending parallel to a line between the front 23 and rear 22. Formed transversely through the post 60 is a round hole 61 in which an adjustment assembly 62 is carried. The adjustment assembly 62 is useful for adjusting the height of the upper collar 11 with respect to the lower collar 12. The post 60 also carries a button assembly 63 just below the adjustment assembly 62 which locks, unlocks, and controls the pitching movement of the arm 13.

In addition to the post 60, the left arm 13 includes a strut 64. The strut 64 and the post 60 are connected to each other at a pitch assembly 65. Referring primarily to FIG. 3 but also to the exploded views of FIGS. 4A and 4B, there are two gear plates 70 and 71 and a base plate 72 captured between the post 60 and the strut 64.

The gear plate 70 is an inner gear plate 70, and the gear plate 71 is an outer gear plate 71. The inner gear plate 70 has a smooth inner face 73 and an outer face 74 formed with a large number of teeth 75. The teeth 75 are oriented radially and extend from the outer edge of the inner gear plate 70 inward, terminating short of a central hole 76 formed entirely through the gear plate 70. The hole 76 is preferably round.

The outer gear plate 71 has an inner face 80 formed with a large number of teeth 81 and an outer face 82 also formed with a large number of teeth 83. The teeth 81 on the inner face 80 are complemental to the teeth 75 on the outer face 74 of the inner gear plate 70. The teeth 81 and 83 are both oriented radially and extend from the outer edge of the outer gear plate 71 inward, terminating short of a central hole 84. The hole 84 corresponds in size and shape to the hole 76 in the inner gear plate 70. While this hole 84 is round, it also includes four circumferentially-spaced apart, radially-oriented slots 85 formed in communication with the hole 84. Pairs of sprung fingers 86 are disposed in the slots 85.

The fingers 86 are formed integrally to the outer gear plate 71 proximate the teeth and have free ends proximate the hole 84. One finger 86 of each pair extends from the teeth 81 radially inward toward the hole 84 and curling beyond the inner face 80, and the other finger 86 of the respective pair extends from the teeth 83 radially inward toward the hole 84 and curling beyond the outer face 82, such that the fingers 86 in each pair curl away from each other proximate the hole 84.

The base plate 72 has an inner face 90, an opposed outer face 91, and a bottom projection 92 carrying a plurality of teeth 93. The inner face 90 is generally smooth but for two vertical rails 94 spaced apart from each other and extending the entire height of the base plate 72 along the inner face 90. The rails 94 flank a central hole 95 which corresponds in size and shape to the holes 84 and 76 in the outer and inner gear plates 71 and 70. On the outer face 91, a hollow cylindrical spindle 96 projects normally outward from the outer face 91, bounding the hole 84. The spindle 96 has an inner face with axial channels 97. Radially-extending holes 98 formed through the spindle 96 are in communication with the channels 97, proximate the outer face 91. But for the spindle 96, the outer face 91 is smooth, configured to receive the smooth inner face 73 of the inner gear plate 70. The bottom projection 92 extends below and outboard of the inner face 90. It includes a plurality of outwardly-projecting teeth 93. These teeth 93 engage with the button assembly 63 to lock, unlock, and control pitching movement of the arm 14.

FIGS. 4A and 4B show an exploded view of the right arm 14, and FIGS. 1-3 show assembled views of the arms 13 and 14. The inner gear plate 70, outer gear plate 71, and the base plate 72 are sandwiched together between the post 60 and the strut 64 to form the pitch assembly 65. The inner gear plate 70, the outer gear plate 71, and the post 60 are all mounted for rotation on and about the spindle 96.

The post 60 includes an inner face 100 on which are formed a large plurality of teeth 101. These teeth 101 are complemental to the teeth 83 on the outer face 82 of the outer gear plate 71. These teeth 101 also extend from the peripheral edge of the post 60 radially inward, terminating short of the hole 61 in the post 60.

In operation, the post 60, the inner gear plate 70, and the outer gear plate 71 fit together in toothed or enmeshed engagement to rotate as a unit. The teeth 75 on the outer face 74 of the inner gear plate 70 mesh with the teeth 81 on the inner face 80 of the outer gear plate 71, and the teeth 83 on the outer face 82 of the outer gear plate 71 mesh with the teeth 101 on the inner face 100 of the post 60. In this way, these three structures are meshed together so as to move and rotate together as a single piece. The post 60 is coupled to the base plate 72 by the inner and outer gear plates 70 and 71, such that movement of the strut 64 imparts movement of the post 60 with respect to the strut 64. Moreover, as the inner gear plate 70, the outer gear plate 71, and the post 60 move, they engage and interact with the base plate 72 to selectively limit and prevent movement.

The base plate 72 has a block 102 projecting outward, normal to the inner face 91. The block 102 is registered above the spindle 96, proximate the top of the base plate 72. The block 102 interacts with two blocks carried on the gear plates 70 and 71 to limit rotational movement of the pitch assembly 65.

The inner gear plate 70 has a block 103 formed on its peripheral edge. The outer gear plate 71 has a similar block 104 formed on its peripheral edge. The two blocks 103 and 104 interact with the block 102 to define the limits of flexion and extension of the strut 64 with respect to the post 60.

Since the block 102 is formed to the base plate 72, it does not move relative to the base plate 72 or the strut 64. It acts as a stop to the other two blocks 103 and 104. A health worker—or even the patient—can adjust the amount of allowable pitch, or range of movement—by setting the blocks 103 and 104 at distances apart from each other. When the blocks 103 and 104 are circumferentially spaced apart from each other, as shown in FIG. 3, they allow a defined or desired range of movement. The patient can pitch his head forward or backward, in flexion and extension, in a first plane of movement until the block 102 on the base plate 72 contacts one of the blocks 103 and 104. When the block 102 contacts the block 103 or 104, further pitching is prevented. The blocks 103 and 104 thus prevent unlimited pitching movement and limit the pitching movement to a defined or desired pitching range of movement, between the blocks 103 and 104.

Markings 105 on the outside of the post 60 (shown in FIGS. 3 and 4A) indicate angular displacements for flexion and extension. The patient or the health worker can thus adjust the gear plates 70 and 71 to change the degree of flexion and extension. The block 102 remains at the top of the markings 105, or at zero degrees. The markings then increment up to preferably sixty degrees of pitch. The health worker can thus precisely set the amount of flexion and extension. To do so, the health worker must slightly disassemble the pitch assembly 65, separate the inner and outer gear plates 70 and 71 from each other slightly, move them until the blocks 103 and 104 register with desired degrees of flexion and extension as indicated by the markings 105, and then move the inner and outer gear plates 70 and 71 back together. To disassemble the pitch assembly 65, the health worker must pull the seat 128 outward along from spindle 96 slightly, to allow the gear plates 70 and 71 to become free of each other.

Other embodiments of the pitch assembly 65 exist. For example, in some embodiments, the pitch assembly 65 includes a ratchet-and-pawl system where a pivoting pawl on the strut 64 engages notches on the post 60 to allow motion in one direction up to either a stop or a detent pin that can drop into different holes corresponding to angular flexion and extension settings. In another embodiment, the pitch assembly 65 includes a series of stacked plates with holes and a removable pin. Aligning different holes sets different angles, and inserting the pin into a hole locks the position. In yet another embodiment, a friction-based clutch hinge includes an adjustable tension dial. The dial is initially fully tightened to immobilize the strut 64 and post 60, and then is gradually loosened to allow more play or movement. Similarly, in some embodiments, the blocks 102, 103, and 104 are replaced or augmented by other limiters, such as adjustable screws that set to contact a flange at a certain angle, or compressible bumpers that allow a bit of cushioning at the end of travel.

In some instances, the health worker will not allow any flexion or extension. Locking out the strut 64 so that it disables pitching movement is accomplished with either the blocks 103 and 104 or with the button assembly 63. In a first way, the health worker adjusts the inner and outer gear plates 70 and 71 so that the blocks 103 and 104 are adjacent to and abut the block 102. This immediately prevents the patient from moving his head forward or backward, because the blocks 103 and 104 immediately confront the fixed block 102.

In a second way, the health worker adjusts the button assembly 63. The button assembly 63 is carried in the post 60 and acts as an additional safety to prevent inadvertent movement of the strut 64 with respect to the post 60. Referring to FIGS. 3-4B, the button assembly 63 includes an outer button 110 and a tooth piece 111. The outer button 110 has a large outer surface formed with two slots 112. The tooth piece 111 has two tabs 113 that fit into those slots 112 to hold the tooth piece 111 to the outer button 110. The outer button 110 is carried on the outer side of the post 60, and the tooth piece 111 is on the inner side of the post 60, such that the tabs 113 also fit through slots 114 in the post 60 to engage with the outer button 110 about the post 60.

The tooth piece 111 has an inner face 115 formed with a set of projecting teeth 116. These teeth 116 engage with the teeth 93 on the bottom projection 92 of the base plate 72. Those teeth 93 are outboard, or radially beyond, the teeth 75, 81, and 83 of the inner and outer gear plates 70 and 71. When the teeth 116 and 93 are engaged with each other, the post 60 is rigidly locked with respect base plate 72 and relative rotational or pitching movement of the post 60 and base plate 72 is prevented or disabled. To disable such movement, the button assembly 63 is slid upward in the slots 114 on the post 60, thereby enmeshing the teeth 116 of the button assembly 63 with the teeth 93 of the base plate 72.

To enable pitching movement, the button assembly 63 is slid down, moving the tabs 113 downward in the slots 114. The teeth 116 are moved downward, out of enmeshed engagement with the teeth 93 of the base plate 72. The base plate 72 can then potentially rotate with respect to the post 60. However, the blocks 103 and 104 must be set apart from the block 102 to enable such movement. If the blocks 103 and 104 are in abutment with the block 102, then sliding the button assembly 653 downward and out of engagement with the teeth 93 of the base plate 72 will not enable pitching movement.

In addition to pitching movement, the arms 13 and 14 are mounted to move in vertical movement. Referring primarily to FIGS. 3, 4A, and 4B, the strut 64 has an upper shoulder 120 (shown only in FIG. 3) and a depending leg 121 pivotally coupled to the shoulder 120. The shoulder 120 is formed integrally to the upper collar 11. A catch on the upper collar 11 is coupled to a rod 122, allowing the leg 121 to rock or tilt laterally with respect to the shoulder 120, thereby allowing the upper collar 11 to laterally tilt or roll with respect to the lower collar 12.

As a brief aside, in other embodiments, the lateral tilt or roll is controlled, adjustable, limitable, or lockable. In one embodiment, the orthosis 10 includes interchangeable stops or shims in the shoulder 120 to reduce or increase the tilt angle, or a small tightening screw that can lock the rod 122 in place (removing lateral play entirely for initial immobilization). In another embodiment, there is a miniature gear or slide mechanism at the shoulder 120. The coupling of the shoulder 120 and leg 121 includes lateral gear teeth and an adjustment similar to the pitch gear to set how much roll is permitted.

Returning to FIGS. 3, 4A, and 4B, below the shoulder 120, the leg 121 has an outer face 123 with two vertical inset channels 124. These receive the complemental rails 94 formed on the inner face 90 of the base plate 72. The channels 124 and rails 94 have a slightly angular cross-section to prevent inadvertent detachment of the base plate 72 from the leg 121. Engagement of the rails 94 within the channels 124 allows the pad to slide up and down with respect to the leg 121.

The upper collar 11 is thus mounted for vertical movement with respect to the lower collar 12 at the arms 13 and 14. The patient or health worker can adjust the vertical height or spacing of the upper collar 11 above the lower collar 12. Flanked by the channels 124, the leg 121 of the strut 64 has a track or slot 125. One side of the slot 125 is formed with linear teeth, forming a rack 126. A pinion 127 is engaged with this rack 126 to raise and lower the upper collar 11. 133

The pinion 127 is part of the adjustment assembly 62. Referring still to FIGS. 4A and 4B primarily, the adjustment assembly includes the pinion 127, a seat 128, and an adjustment knob 129.

The seat 128 has an enlarged head 130 and a short stub 131 projecting inward from the head 130. The head 130 has a recessed socket and a bore formed therein. The seat 128 is carried in the spindle 96 projecting from the base plate 72. When the pitch assembly 65 is assembled, the head 130 of the adjustment assembly 62 fits against the outer face of the post 60 and the stub 131 fits into the spindle 96, confined therein. At the end of the stub 131 are two flanges 132 extending outward in opposed directions; these flanges 132 snap into the holes 98 at the base of the spindle 96 to hold and secure the seat 128 in the spindle 96. The seat 128 acts as a cap holding the post 60 in against the inner and outer gear plates 70 and 71, the base plate 72, and the strut 64.

The adjustment knob 129 is carried in the seat 128 for rotation. The adjustment knob 129 has a knob 133 and a slender shank or pin 134 extending from the knob 133. The knob 133 seats into the seat 128 and is carried therein freely for rotation. The pinion 127 is secured to the pin 134, opposite the seat 128 from the knob 133. The rack includes a short shank 135 and a pinion gear 136.

When the adjustment assembly 62 is carried in the spindle 96, the pinion gear 136 is engaged with the rack 126 in the strut 64, and the knob 133 is on the outside of the post 60 and is available to be turned. Rotation of the knob 133 imparts rotation to the pinion gear 136 along the rack 126. This imparts vertical movement to strut 64 with respect to the post 60. Rotation in a first direction causes the strut 64 to raise with respect to the post 60, and rotation in a second direction causes the strut 64 to lower with respect to the post 60.

As a brief aside, in other embodiments of the orthosis 10, vertical movement is accomplished in other ways. For example, in an embodiment, the orthosis 10 includes a discrete ratchet lift in which fine pitch teeth and a positive over center pawl hold a set lift against a head load. In another embodiment, a micro screw jack assembly includes a lead screw with an external thumb dial or hex drive that raises the base plate 72 relative to the post 60 in sub millimeter increments. An internal torque limiter clutch caps the applied force to a prescribed maximum and slips safely beyond that threshold. In yet another embodiment, an elastic constant force module assembly includes a preloaded spring or constant force element that provides a near constant upward bias. A calibration collar or preload cam adjusts baseline tension, and the assembly may be paired with a short throw screw to “trim” height. In yet still another embodiment, a pneumatic/hydraulic cell assembly includes a compact inflatable or fluid bladder positioned in line with either or both arms 13 and 14 to generate lift, and a check valve and a relief valve limit pressure and allow quick release.

Returning to the main discussion, as noted above, the left and right arms 13 and 14 are identical and mirror opposites. The upper collar 11 is mounted to those arms 13 and 14. Referring to FIGS. 1 and 2 primarily, the upper collar 11 is a wide base for supporting the patient's head. It distributes the weight and pressure of the head. This helps prevent hot spots and sores on the jaw or back of head and also helps prevent degradation of the upper collar 11 itself.

The upper collar 11 is preferably formed from two shells: a left shell 140 and a right shell 141. In other embodiments, the upper collar 11 is formed from anterior and posterior shells, and in other embodiments, there are other shells or other numbers and configurations. The two shells 140 and 141 here are identical and mirror opposites. As such, reference is made herein to either or both shells without distinction, and the same reference characters are used to identify the same structural elements and features in the shells 140 and 141. Where the description refers to one of the shells 140 or 141, the reader will understand that the description applies equally to the other of the shells 141 or 140 unless specifically noted.

The shell 141 has a rear 142, a front 143, and a body 144 extending therebetween. The body 144 is thin and wide. It has a width extending between an inner edge 145 and an outer edge 146, and it has an inner or upper surface 147 and an opposed outer or lower surface 148. The width between the inner and outer edges 145 is greater than the thickness between the upper and lower surfaces 147 and 148. Very roughly, and without limitation, the width of the body 144 may preferably be between one and two inches, though in other embodiments it may be more or less. Because the orthosis 10 is custom constructed for a patient, some patients may need or want a narrower or wider upper collar 11, and some health workers may order upper collars 11 of different sizes for specific needs or functions. Regardless, generally, the width of the upper collar 11 is much greater than its thickness.

The two rears 142 of the left and right shells 140 and 141 abut each other and are coupled to each other at a hinge 150, as shown in FIG. 2. The hinge 150 includes a left leaf 151 on the left shell 140 and a right leaf 152 on the right shell 141. The leaves 151 and 152 engage with each other to form a knuckle 153 in which a pin 154 sits. The pin 154 is registered with the axis 35 of the other pin 34 in the hinge 30 of the lower collar 12.

At the front, the left and right shells 140 and 141 also couple to each other. Referring to FIG. 1 again, the left and right shells 140 and 141 connect at their fronts 143 with a clasp 160. In the embodiment shown herein, the clasp 160 includes a low dome 161 with a central hole 162. The dome 161 is fixed to the left shell 140 but extends partially beyond the front 143 of the shell 140, such that the hole 162 is disposed half over the shell 140 and half off the shell 140. The right shell 141 carries a tongue 163 which projects off the front 143 of the right shell 141 toward the left shell 140. The tongue 163 terminates in an enlarged head 164 which is sized and shaped to snap into the hole 162 of the left shell 140. When the fronts 143 of the shells 140 and 161 are brought together, the tongue 163 moves into the dome 161 and the head 164 snaps into the hole 162, thereby securing the clasp 160. The clasp 160 will not come unbuckled unless a person depresses the head 164 from the hole 162, allowing the tongue 163 to be removed from the dome 161 and the two shells 140 and 161 to be separated from each other.

The left and right shells 140 have a variety of configurations. In some embodiments, the shells 140 are just bands, in which the inner and outer edges 145 and 146 are roughly parallel along their entire lengths. This is shown by the broken line in FIG. 1 and a visible solid line in FIG. 2.

In some situations, the patient needs lateral support of his head. In those situations, the upper collar 11 includes the left and right occipital wings 170 and 171. The wings 170 and 171 are identical and mirror opposites. As such, reference is made herein to either wing without distinction, and the same reference characters are used to identify the same structural elements and features in the wings 170 and 171. The wing 171 extends upward from the outer edge 146 of the left shell 140.

The wing 171 has an arcuate perimeter edge 172 that extends upward from the outer edge 146, curves around, and returns back to the outer edge 146 in arc fashion. The wing 171 has a concave shape inward to cradle the sides of the patient's head. The wing 171 preferably carries padding 173 on its inner face, and in some embodiments, shims or padding 173 of different thicknesses are used to provide different levels of lateral support for the head. When patients are in need of lateral tilt or roll support for their head, the orthosis is created with either or both of the wings 171. In some cases, only one wing 171 or the other may be needed, while in some cases, both may be needed. The wing 171 is formed monolithically to the body 144 of the upper collar 11. For example, when a patient has torticollis, his head may tilt to the side, and his orthosis 10 may require a heavily padded wing on one side or the other.

In some cases, more support may be required. The embodiment of the orthosis 10 shown here includes a posterior shell 175 formed to the body 144. In this embodiment, the posterior shell 175 includes left and right pieces, but in other embodiments, it is formed from a single piece. In this embodiment, the posterior shell 175 extends upward from a base 176 at the rears 142 of the left and right shells 140 and 141 to a top 177. The posterior shell 175 is preferably rigidly formed to the body 144 and is constructed from a material or combination of materials having rigid and resilient material characteristics. The posterior shell 175 supports the back of the patient's head and in some embodiments biases the head forward.

The posterior shell 175 includes tabs 178 at the top 177 which project forwardly on both sides of the posterior shell 175. These tabs 178 wrap around and support the side of the patient's head.

In some embodiments, the posterior shell 175 also incorporates a strap 180. The strap 180 is secured near the top 177 of the posterior shell 175 with loops 181 on the outer surface of the posterior shell 175. The strap 180 wraps around the top of the patient's head to hold it against the posterior shell 175. In some embodiments, the strap 180 is elastic, and in some embodiments it has an adjustable length.

The orthosis 10 is custom-created and custom-fit to a patient. FIGS. 5A-7B illustrate an orthosis 10 embodiment in different stages of operation and movement. FIGS. 5A and 5B illustrate the orthosis 10 moving through a range of pitching movement in a sagittal plane of movement illustrated by the broken lines marked with the reference character S in FIG. 5A. The arms 13 and 14—and the pitch assemblies 65 in each arm—enable this pitching movement of the upper collar 11 with respect to the lower collar 12. Preferably, the patient moves the upper collar 11 only as prescribed or set by the health worker. The blocks 103 and 104 are stops that interact with the block 102 to limit this pitching movement however. The health worker can adjust the position of the blocks 103 and 104 to increase or decrease the defined or desired range of pitching movement. The pitching movement occurs about an axis of rotation, marked with the reference character A. The spindle 96 defines the axis A. This pitching movement is unicentric, but in other embodiments, the pitching movement is polycentric and occurs around two or more axes of rotation.

As shown in FIGS. 5A and 5B, the health worker has set a fairly large range of movement. The “rear” block 103 is positioned at roughly sixty or seventy degrees in the posterior direction, and the “front” block 104 is positioned at roughly sixty or seventy degrees in the anterior direction, so that there is roughly one-hundred twenty to one-hundred forty degrees in the range of pitching movement available to the patient wearing this orthosis. FIG. 5A shows the orthosis 10 pitched fully forward, in the anterior direction. The upper collar 11 is pivoted forwardly and downwardly, and so the strut 64 of the right arm 14 pitches forwardly relative to the post 60. The left arm 13 also pitches forwardly, but because it is not visible in the views of FIGS. 5A and 5B, reference is made only with respect to the right arm 14.

The block 102 is fixed to the strut 64 and pivots forwardly when the upper collar pivots forwardly. This moves the block 102 toward the front block 104. When it contacts the front block 104, it stops, and so the strut 64 stops, and so the upper collar 11 stops. Interaction of the blocks 102 and block 104 stops forward pitching movement of the upper collar 11 with respect to the lower collar 12.

In FIG. 5B, the orthosis 10 is now pitched fully backward, in the posterior direction. The upper collar 11 is pivoted rearwardly and downwardly, and so the strut 64 pitches rearwardly relative to the post 60. The block 102, fixed to the strut 64, pivots rearwardly, too. When it contacts the rear block 103, it stops, and so the strut 64 stops, and so the upper collar 11 stops. Interaction of the blocks 102 and block 104 stops rearward pitching movement of the upper collar 11 with respect to the lower collar 12.

FIGS. 6A and 6B illustrate the orthosis moving through a defined or desired range of vertical movement in a frontal plane of movement illustrated by the broken lines marked with the reference character F in FIG. 6A. The arms 13 and 14, and the pitch assemblies 65 in each arm, enable this vertical movement of the upper collar 11 with respect to the lower collar 12. Preferably, the patient moves the upper collar 11 only as prescribed or set by the health worker, to provide the proper amount of traction to the patient.

The upper collar 11 moves with respect to the lower collar 12 between a raised position, shown in FIG. 6A, and a lowered position, shown in FIG. 6B. To move the upper collar 11 vertically, the health worker rotates the knob 133. The knob 133 is fixed on the pin 134 (shown in FIGS. 4A and 4B), the pinion 127 is fixed on the pin 134. Rotation of the knob 133 thereby imparts rotation to the pin 127, which is meshingly engaged with the rack 126. Rotation of the knob 133 in one direction, such as clockwise, causes the pinion gear 136 to rotate clockwise and drive the rack downward 126. The rack 126 is integral or attached to the strut 64, and so the strut 64 is driven downward with respect to the post 60. The base plate 72 and the strut 64 move relatively to each other, with the rails 94 of the base plate 72 sliding in the channels 124 of the strut 64. This causes the upper collar 11 to lower vertically with respect to the lower collar 12.

Rotation of the knob 133 in another direction, such as counter-clockwise, causes the pinion gear 136 to rotate counter-clockwise and drive the rack 126 upward, thereby driving the strut 64 upward as well. This causes the upper collar 11 to rise vertically with respect to the lower collar 12.

Preferably, the health worker adjusts the knobs 133 on both left and right arms 13 and 14, so that the two sides of the upper collar 11 rise or lower together. The health worker can make both gross and fine adjustments, rotating the knobs 133 in small increments to achieve very small and non-discrete adjustments. This maintains the upper collar 11 in a relatively horizontal parallel arrangement with the lower collar 12.

As a brief aside, in some embodiments of the orthosis 10, there are safety mechanisms for setting and maintaining traction at a desired level. In one embodiment, the orthosis 10 includes a bidirectional scale, which includes a graduated height scale co-located with channels and a force indicator (spring scale, calibrated torque window on screw, pressure gauge for) indicating approximate axial distraction force. In another embodiment, a force-limiting clutch, or torque limiter (or relief valve) provides a hard ceiling on traction, acting as a fail safe against over distraction. In yet another embodiment, the orthosis 10 includes a timed release. An optional time based detent or electronic timer cues or triggers gradual return to baseline after a clinician set interval (e.g., cyclic traction).

Returning to the main discussion, in some circumstances, the health worker may desire to set the left and right arms 13 and 14 at different heights. The health worker need only adjust the left and right arms 13 and 14 independently as desired. When one arm 13, 14 has a different height than the other arm 13, 14, the arms 13 and 14 hinge slightly. Referring to the view shown in FIGS. 6A and 6B, the right arm 14 includes the upper shoulder 120 and the strut 64 which is pivotally coupled to the shoulder 120. The shoulder 120 is fixed to the upper collar 11 and is preferably integrally formed to the body 144 thereof. The shoulder 120 has a catch, to which the rod 122 in the strut 64 is coupled for pivotal movement.

When the health worker adjusts the left and right arms 13 and 14 at different heights, the upper collar 11 rolls to one side. As the upper collar 11 rolls, the strut 64 pivots on the shoulder 120 to enable and accommodate that rolling movement. In some embodiments, the arms 13 and 14 include a micro damper to dissipate energy on sudden unloading between the strut 64 and the post 60. In other embodiments, the arms 13 and 14 include a quick-release or single action button to collapse the arms 13 and 14, returning them to a baseline height rapidly if discomfort occurs.

FIGS. 7A and 7B illustrate the orthosis 10 moving through a defined or desired range of rotational movement in a transverse plane of movement illustrated by the broken lines marked with the reference character T. The arms 13 and 14 enable rotational movement of the upper collar 11 with respect to the lower collar 12. Preferably, the patient moves the upper collar 11 only as prescribed or set by the health worker. The set screws 55 are stops that interact with the arms 13 and 14 to limit this rotational movement, however. The health worker can adjust the position of the set screws 55 to increase or decrease the range of rotational movement.

When the set screws 55 are removed, the upper collar 11 is enabled to move through a full range of rotational movement parallel to the transverse plane T. That includes the right arm 14 moving along the track 50 entirely between the rear and front ends 53 and 54 of the track 50. As shown in FIGS. 7A and 7B, the health worker has set a large range of movement; the set screws 55 are positioned at, or just inboard of, the rear and front ends 53 and 54, enabling movement of the right arm 14 along nearly the full extent of the track 50. The left track 50 is similarly arranged but is less visible in FIGS. 7A and 7B, which is why reference is made only to the right side of the orthosis 10 here.

The bottom 56 of the arm 14 is carried in the track 50 and can slide freely therein through a rotational range of movement. This enables the patient to turn his head left and right, when the set screws 55 so allow. As in FIGS. 7A and 7B, the set screws 55 are widely set off from the bottom 56 of the arm 14, thereby making the defined range of rotational movement quite large.

If desired, the health worker can position the set screws 55 as shown in FIGS. 1-3, where the set screws 55 are immediately adjacent the bottom 56 of the arm 14. In that arrangement, the right arm 14 is prevented from rotational movement completely. With the right arm 14 prevented from movement, the upper collar 11 cannot move in rotational movement, either. The health worker must move the set screws 55 around both the left and right arms 13 and 14 to enable rotational movement.

In some embodiments, the orthosis 10 includes electronics for monitoring and guidance. Small, unobtrusive sensors capture useful data: for instance, an angle sensor in the pitch assembly 65 (like a potentiometer or magnetic encoder on the spindle 96) directly measure the degree of flexion or extension the patient is achieving. In an embodiment, a gyroscope/accelerometer (IMU) module on the ring tracks overall head orientation and movement frequency. In another embodiment, strain gauges on the arms measure how much force the patient exerts against the stops (useful to gauge muscle effort). In yet another embodiment, pressure sensors embedded in padding monitor skin pressure and ensure it stays below risky levels.

This data can be used in several ways. A basic use is simply recording compliance: the orthosis 10 logs each time the patient moves beyond a certain small threshold, indicating they are doing neck exercises or moving too much. The data shows if the patient is adhering to restrictions. This informs health workers during follow-up.

Additionally, the data enables real-time feedback. In embodiments, the orthosis 10 connects via Bluetooth to a smartphone app or a hospital monitoring system. The app guides the patient through prescribed movements within a safe range of movement. Sensors confirm the motion and ensure it is done slowly correctly. The app then signals to an electronic actuator in the orthosis 10 to increase the allowed range if appropriate in the next phase of treatment.

In other embodiments, orthosis 10 simply has indicator LEDs, an audible alarm, or a haptic device to alert when approaching a range of movement limit to encourage the patient to stop. Preferably, in these embodiments, the orthosis 10 includes memory to track usage time to improve compliance with wearing schedules.

FIGS. 8-12B illustrate another exemplary embodiment of a cervical collar orthosis 210 (hereinafter, “orthosis 210”). The orthosis 210 is similar to the orthosis 10 and has many of the same structural elements and features as the orthosis 10. As such, where the description refers to the same structural elements and features, it adopts the same reference characters as those used in connection with the orthosis 10, but marks them with a prime symbol (“′”) so as to distinguish them from reference characters used in connection with the orthosis 10.

The orthosis 210 includes a cranial support ring or upper collar 11′, a lower base or lower collar 12′, and left and right armatures or support arms 213 and 214, respectively, extending between the upper and lower collars 11′ and 12′. The upper collar 11′ is held in vertically spaced relation with respect to the lower collar 12′ by the arms 213 and 214. Those arms 213 and 214 set the height of the upper collar 11′ above the lower collar 12′, establish the pitch (the flexion and extension) of the upper collar 11′ with respect to the lower collar 12′, determine the roll of the upper collar 11′ with respect to the lower collar 12′, and control the rotation of the upper collar 11′ with respect to the lower collar 12′. The upper and lower collars 11′ and 12′ are entirely customizable. The lower collar 12′ is formed to match the features and curvature of a patient's upper torso and neck area, and the upper collar 11′ is formed to match exactly the features and curvature of a patient's neck, chin, and head. Moreover, the placement or location of the left and right arms 213 and 214 along the upper and lower collars 11′ and 12′ can be changed to fit the patient's anatomy, functional requirement, and specific needs.

The lower collar 12′ is a wide base for the orthosis 210. It distributes the weight and pressure of the orthosis 210 across the sternum, the upper back, the clavicles, trapezius muscles, and other upper portions of the torso. This helps prevent hot spots and sores on the body and also helps prevent degradation of the orthosis 210 itself.

The lower collar 12′ is preferably formed from two shells. In the embodiment shown in these drawings, those shells include a left shell 20′ and a right shell 21′. In other embodiments, the shells are anterior and posterior shells, and in other embodiments, there are other shells of other numbers and configurations. Here, the two shells 20′ and 21′ are identical and mirror opposites. As such, reference is made herein to either shell without distinction, and the same reference characters are used to identify the same structural elements and features in the shells 20′ and 21′. Where the description refers to one of the shells 20 or 21, the reader will understand that the description applies equally to the other of the shells 21′ or 20′ unless specifically noted. The shells 20′ and 21′ are shown in FIGS. 1 and 2.

The shell 21′ has a rear 22′, a front 23′, and a body 24′ extending therebetween. The body 24′ is thin and wide. It has a width extending between an inner edge 25′ and an outer edge 26′, and it has an upper surface 27′ and an opposed lower surface 28′. The width between the inner and outer edges 25′ and 26′ is much greater than the thickness between the upper and lower surfaces 27′ and 28′. Very roughly, and without limitation, the width of the body 24′ may preferably be between one and three inches, though in other embodiments it may be more or less. Because the orthosis 210 is custom constructed for a patient, some patients may need or want a narrower or wider lower collar 12′, and some health workers may order lower collars 12 of different sizes for specific needs or functions. Regardless, generally, the width of the lower collar 12′ is much greater than its thickness.

The two rears 22′ of the left and right shells 20′ and 21′ abut each other and are coupled to each other at a hinge 30′. The hinge 30′ is shown in FIG. 11A and not discussed in detail here; its structure is preferably the same as that of the hinge 30. At the fronts 23′, the left and right shells 20′ and 21′ also couple to each other. Referring to FIG. 1 again, the left and right shells 20′ and 21′ connect at their fronts 23 with a clasp 40′. Like the hinge 30′ the clasp 40′ is identical to the clasp 40, and this discussion need not repeat the description of the clasp 40. The lower surfaces 28′ of the left and right shells 20′ and 21′ carry padding elements 45′ and 46′. Their description is also not repeated here.

The left and right arms 213 and 214 are coupled to the lower collar 12′ for movement. The left and right arms 213 and 214 are identical and mirror opposites. As such, reference is made herein to either or both arms without distinction, and the same reference characters are used to identify the same structural elements and features in the arms 213 and 214. Where the description refers to one of the arms 13 or 14, the reader will understand that the description applies equally to the other of the arms 14 or 13 unless specifically noted. The arms 213 and 214 are shown in FIG. 8 and in exploded view in FIGS. 9A and 9B.

The right shell 21′ includes a track 50′ formed on the outer surface 28′. The track 50′ is arcuate and extends a majority of the length of the right shell 21′ between the rear 22′ and the front 23′. The track 50′ projects upward. It has an upstanding sidewall 51′ that terminates in a top formed with an elongate slot 52′ extending between a rear end 53′ and a front end 54′. A bottom of the right arm 214 is carried in the track for sliding, reciprocating movement along the track 50′ between the rear and front ends 53′ and 54′. Threaded set screws 55′ threadably engage with the slot 52 to tighten and loosen in the slot 52′. The screws 55′ can be loosened, slid along the track 50′ to a desired position, and then tightened and secured in the desired position. In this way, the screws 55′ are stops which limit a range of movement of the right arm 214 in the track 50′ to between the screws 55′ rather than between the full extent of the track 50′ itself.

The right arm 214 includes a base post, or post 220, with a tongue carried in the track 50′. The tongue is enlarged such that it cannot be removed from the slot 52′. The right arm 214 is thus mounted for reciprocal movement in the track 50′.

The post 220 projects upwardly from the lower collar 12′ at approximately two-thirds the distance from the front 23′ to the rear 22′ of the lower collar 12′. The post 220 is slender, having opposed major faces directed inward toward the patient and outward away from the patient. The post 220 has a peripheral edge that arcuately bends upward from and then downward to the lower collar 12′, roughly along a line extending parallel to a line between the front 23′ and rear 22′. Formed transversely through the post 220 is a round hole 221 in which an adjustment assembly 222 is carried. The adjustment assembly 222 is useful for adjusting the height of the upper collar 11′ with respect to the lower collar 12′. The post 220 also carries a button assembly 223 just below the adjustment assembly 222 which locks, unlocks, and controls the pitching movement of the arm 213.

In addition to the post 220, the left arm 213 includes a strut 224. The strut 224 and the post 220 are connected to each other at a pitch assembly 225. Referring primarily to the exploded views of FIGS. 9A and 9B, there are two gear plates 230 and 231 and a base plate 229 captured between the post 220 and the strut 224.

The gear plate 230 is an inner gear plate 230, and the gear plate 231 is an outer gear plate 231. The inner gear plate 230 has a smooth inner face 232 and an outer face 74 formed with a large number of teeth 234. The teeth 234 are oriented radially and extend from the outer edge of the inner gear plate 230 inward, terminating short of a central hole 235 formed entirely through the gear plate 230. The hole 235 is preferably round.

The outer gear plate 231 has an inner face 240 formed with a large number of teeth 241 and an outer face 242 also formed with a large number of teeth 243. The teeth 241 on the inner face 240 are complemental to the teeth 234 on the outer face 74 of the inner gear plate 230. The teeth 241 and 83 are both oriented radially and extend from the outer edge of the outer gear plate 231 inward, terminating short of a central hole 244. The hole 244 corresponds in size and shape to the hole 235 in the inner gear plate 230. While this hole 244 is round, it also includes four circumferentially-spaced apart, radially-oriented slots 245 formed in communication with the hole 244. Pairs of sprung fingers 246 are disposed in the slots 245.

The fingers 246 are formed integrally to the outer gear plate 231 proximate the teeth and have free ends proximate the hole 244. One finger 246 of each pair extends from the teeth 241 radially inward toward the hole 244 and curling beyond the inner face 240, and the other finger 246 of the respective pair extends from the teeth 243 radially inward toward the hole 244 and curling beyond the outer face 242, such that the fingers 246 in each pair curl away from each other proximate the hole 244.

The base plate 229 has an inner face 250, an opposed outer face 251, and a bottom projection 252 carrying a plurality of teeth 253. The inner face 250 includes two vertical rails 254 spaced apart from each other and extending the entire height of the base plate 229 along the inner face 250. The rails 254 flank a central hole 255 which corresponds in size and shape to the holes 235 and 244 in the outer and inner gear plates 231 and 230. On the outer face 251, a hollow cylindrical spindle 256 projects normally outward from the outer face 251, bounding the hole 244. The spindle 256 has an inner face with axial channels 257. Radially-extending holes 258 formed through the spindle 256 are in communication with the channels 257, proximate the outer face 251. The bottom projection 252 extends below and outboard of the inner face 250. The outwardly-projecting teeth 253 on the bottom projection 252 engage with the button assembly 223 to lock, unlock, and control pitching movement of the arm 214.

FIGS. 9A and 9B show an exploded view of the right arm 214, and FIG. 8 shows an assembled view of the arms 213 and 214. The inner gear plate 230, outer gear plate 231, and the base plate 229 are sandwiched together between the post 220 and the strut 224 to form the pitch assembly 225.

The post 220 includes an inner face 260 on which are formed a large plurality of teeth 261. These teeth 261 are complemental to the teeth 243 on the outer face 242 of the outer gear plate 231. These teeth 261 also extend from the peripheral edge of the post 220 radially inward, terminating short of the hole 221 in the post 220.

In operation, the post 220, the inner gear plate 230, and the outer gear plate 231 fit together in toothed or enmeshed engagement to rotate as a unit. The teeth 234 on the outer face 233 of the inner gear plate 230 mesh with the teeth 241 on the inner face 240 of the outer gear plate 231, and the teeth 243 on the outer face 82 of the outer gear plate 231 mesh with the teeth 261 on the inner face 260 of the post 220. In this way, these three structures are meshed together so as to move and rotate together as a single piece. As they move, they engage and interact with the base plate 229 to selectively limit and prevent movement.

The base plate 229 has a block 262 projecting outward, normal to the inner face 260. The block 262 is registered above the spindle 256, proximate the top of the base plate 229. The block 262 interacts with two blocks carried on the gear plates 230 and 231 to limit rotational movement of the pitch assembly 225.

The inner gear plate 230 has a block 263 formed on its peripheral edge. The outer gear plate 231 has a similar block 264 formed on its peripheral edge. The two blocks 263 and 264 interact with the block 262 to define the limits of flexion and extension of the strut 224 with respect to the post 220.

Since the block 262 is formed to the base plate 229 it does not move relative to the base plate 229 or the strut 224. It acts as a stop to the other two blocks 263 and 264. A health worker—or even the patient—can adjust the amount of allowable pitch, or range of movement—by setting the blocks 263 and 264 at distances apart from each other. When the blocks 263 and 264 are circumferentially spaced apart from each other, they allow a defined range of movement. The patient can pitch his head forward or backward, in flexion and extension, in a first plane of movement until the block 262 on the base plate 229 contacts one of the blocks 263 and 264. When the block 262 contacts the block 263 or 264, further pitching is prevented. The blocks 263 and 264 thus prevent unlimited pitching movement and limit the pitching movement to a desired pitching range of movement, between the blocks 263 and 264.

Markings 265 on the outside of the post 220 (shown in 9A) indicate angular displacements for flexion and extension. The patient or the health worker can thus adjust the gear plates 230 and 231 to change the degree of flexion and extension. The block 262 remains at the top of the markings 265, or at zero degrees. The markings then increment up to preferably seventy degrees of pitch. The health worker can thus precisely set the amount of flexion and extension. To do so, the health worker must slightly disassemble the pitch assembly 225, separate the inner and outer gear plates 230 and 231 from each other slightly, move them until the blocks 263 and 264 register with desired degrees of flexion and extension as indicated by the markings 265, and then move the inner and outer gear plates 230 and 231 back together.

In some instances, the health worker will not allow any flexion or extension. Locking out the strut 224 so that it disables pitching movement is accomplished with either the blocks 263 and 264 or with the button assembly 223. In a first way, the health worker adjusts the inner and outer gear plates 230 and 231 so that the blocks 263 and 264 are adjacent to and abut the block 262. This immediately prevents the patient from moving his head forward or backward, because the blocks 263 and 264 immediately confront the fixed block 262.

In a second way, the health worker adjusts the button assembly 223. The button assembly 223 is carried in the post 220 and acts as an additional safety to prevent inadvertent movement of the strut 224 with respect to the post 220. Referring still to FIGS. 9A and 9B, the button assembly 223 includes an outer button 270 and a tooth piece 271. The outer button 270 has a large outer surface formed with two slots 272. The tooth piece 271 has two tabs 273 that fit into those slots 272 to hold the tooth piece 271 to the outer button 270. The outer button 270 is carried on the outer side of the post 220, and the tooth piece 271 is on the inner side of the post 220, such that the tabs 273 also fit through slots 274 in the post 220 to engage with the outer button 270 about the post 220.

The tooth piece 271 has an inner face 275 formed with a set of projecting teeth 276. These teeth 276 engage with the teeth 253 on the bottom projection 252 of the base plate 229. Those teeth 253 are outboard, or radially beyond, the teeth 234, 243, and 253 of the inner and outer gear plates 230 and 231. When the teeth 276 and 253 are engaged with each other, the post 220 is rigidly locked with respect base plate 229 and relative rotational or pitching movement of the post 220 and base plate 229 is prevented or disabled. To disable such movement, the button assembly 223 is slid upward in the slots 274 on the post 220, thereby enmeshing the teeth 276 of the button assembly 223 with the teeth 253 of the base plate 229.

To enable pitching movement, the button assembly 223 is slid down, moving the tabs 273 downward in the slots 274. The teeth 276 are moved downward, out of enmeshed engagement with the teeth 253 of the base plate 229. The base plate 229 can then potentially rotate with respect to the post 220. However, the blocks 263 and 264 must be set apart from the block 262 to enable such movement. If the blocks 263 and 264 are in abutment with the block 262, then sliding the button assembly 2253 downward and out of engagement with the teeth 253 of the base plate 229 will not enable pitching movement.

In addition to pitching movement, the arms 213 and 214 are mounted to move in vertical movement. The strut 224 has an upper shoulder 280 (shown in FIG. 8) and a depending leg 281 pivotally coupled to the shoulder 280 (shown in FIGS. 9A and 9B). The shoulder 280 is formed integrally to the upper collar 11′. A catch on the upper collar 11′ is coupled to a rod 282, allowing the leg 281 to rock or tilt laterally with respect to the shoulder 280, thereby allowing the upper collar 11′ to laterally tilt or roll with respect to the lower collar 12′.

Below the shoulder 280, the leg 281 has an outer face 283 with two vertical inset channels 284. These receive the complemental rails 254 formed on the inner face 250 of the base plate 229. The channels 284 and rails 254 have a slightly angular cross-section to prevent inadvertent detachment of the base plate 229 from the leg 281. Engagement of the rails 254 within the channels 284 allows the pad to slide up and down with respect to the leg 281.

The upper collar 11′ is thus mounted for vertical movement with respect to the lower collar 12′ at the arms 213 and 214. The patient or health worker can adjust the vertical height or spacing of the upper collar 11′ above the lower collar 12′.

Flanked by the rails 254, the base plate 229 includes a plurality of sockets 285 arranged vertically along the base plate 229. Two pegs—a top peg 286 and a bottom peg 287—engage with these sockets 285. A health worker applies the pegs 286 and 287 through a large central slot 288 in the center of the strut 224 and then into the sockets 285 to adjust the vertical range of movement of the orthosis 10. The slot 288 has a top 290 and an opposed bottom 291. When the pegs 286 and 287 interact with the top 290 and bottom 291, they prevent further vertical movement in that direction.

The pegs 286 and 287 each have handles 292 on one side of a flange 293, and a stub 294 on the other side of the flange 293. The health worker can grasp the handle 292 and push each peg 286, 287 into a socket 285 until the flange 293 is seated against an inside face 295 of the strut 224. When so seated, the stub 294 protrudes through the slot 288 and into the sockets 285.

As noted above, the left and right arms 213 and 214 are identical and mirror opposites. The upper collar 11′ is mounted to those arms 213 and 214. Referring to FIGS. 1 and 2 primarily, the upper collar 11′ is a wide base for supporting the patient's head. It distributes the weight and pressure of the head. This helps prevent hot spots and sores on the jaw or the back of head and also helps prevent degradation of the upper collar 11′ itself.

The upper collar 11′ is preferably formed from two shells: a left shell 140′ and a right shell 141′. In other embodiments, the upper collar 11′ is formed from anterior and posterior shells, and in other embodiments, there are other shells or other numbers and configurations. The two shells 140′ and 141′ here are identical and mirror opposites. As such, reference is made herein to either or both shells without distinction, and the same reference characters are used to identify the same structural elements and features in the shells 140′ and 141′. Where the description refers to one of the shells 140′ or 141′, the reader will understand that the description applies equally to the other of the shells 141 or 140 unless specifically noted.

The shell 141′ has a rear 142′, a front 143′, and a body 144′ extending therebetween. The body 144′ is thin and wide. It has a width extending between an inner edge 145′ and an outer edge 146′, and it has an inner or upper surface 147′ and an opposed outer or lower surface 148′. The width between the inner and outer edges 145′ is greater than the thickness between the upper and lower surfaces 147′ and 148′. Very roughly, and without limitation, the width of the body 144′ may preferably be between one and two inches, though in other embodiments it may be more or less. Because the orthosis 210 is custom constructed for a patient, some patients may need or want a narrower or wider upper collar 11′, and some health workers may order upper collars 11′ of different sizes for specific needs or functions. Regardless, generally, the width of the upper collar 11′ is much greater than its thickness.

The two rears 142′ of the left and right shells 140′ and 141′ abut each other and are coupled to each other at a hinge 150′. At the front, the left and right shells 140′ and 161 also couple to each other with a clasp 160′. The hinge 150′ and clasp 160′ are identical to the hinge 150 and clasp 160, and no further description of them is necessary.

Like the left and right shells 140 and 141, the left and right shells 140′ and 141′ have a variety of configurations, such as including a band, wings 170′ and 171′, a posterior shell 175′, tabs 178′, and a strap 180′. Those structures are identical to the corresponding structures in the orthosis 10 and no further description of them is necessary.

The orthosis 210 moves in the same sagittal, transverse, and frontal planes of movement that the orthosis 10 does, and the reader will readily appreciate the relative ranges of movement from that previous description. However, the orthosis 210 includes structures and features not present in the orthosis 10. The orthosis 210 includes bands to provide assistance or resistance to movement of the upper collar 11′ relative to the lower collar 12′ in several directions of movement. The bands are preferably elastic, and they impart a bias between the post 220 and the strut 224 to either assist or resist movement by loading or unloading forces in several directions of movement. This can aid a patient in moving or can force a patient to work therapeutically through a range of movement.

Turning to FIGS. 9A and 9B, the post 220 includes upper pegs 300 and lower pegs 301 on each side of the post 220. These pegs 300 and 301 project integrally and laterally from the sides of the post 220. There are four bands in this embodiment: front and rear long bands 302 and front and rear short bands 303. All four of the bands 302 and 303 are attached to the pegs 300 and 301 on the post 220. The bands then extend to the strut 224. Upper pegs 304 extend laterally out from both sides of the strut 224 proximate its top, and a single long peg 305 is at the bottom of the strut 224.

With attention to FIG. 10A, the long band 302 extends from the lower peg 301 on the post 220 to the upper peg 304 on the strut 224, on the front of the right arm 214. Another long band 302 extends similarly on the back of the right arm 213. The short band 303 extends from the upper peg 300 on the post 220 to the lower peg 305 on the strut 224, on the front of the right arm. Another short 30 extends similarly on the back of the right arm 214. The left arm 213 also has a similar arrangement.

When the patient pitches his head and moves the orthosis 210 in pitching movement between the two positions shown in FIGS. 10A and 10B, the bands can either assist or resist this movement. For example, if the health worker desires to assist the patient in pitching the head forward, he can apply the bands 302 and 303 to only the front of the left and right arms 213 and 214 and place no bands on the back of the arms 213 and 214. The bands then provide an assistive force when the patient moves his head forward and down. Alternatively, to assist the patient in pitching head backward, the health worker would place the bands 302 and 303 only on the pegs on the back of the arms 213 and 214.

Similarly, the bands can be used in vertical movement. During vertical movement, as shown in FIGS. 11A and 11B, the long band 302 stretches and recoils considerably. The long band 302 provides a resistive force in the vertical direction. It pulls the strut 224 downward toward the post 220, creating axial compression or axial loading of the upper collar 11′ against the lower collar 12′. On the other hand, the short band 303 provides an assistive force in the vertical direction. It pulls the bottom of the strut 224 upward from the upper peg 300 of the post 220, creating axial decompression or axial unloading of the upper collar 11′ with respect to the lower collar 12′.

The bands 302 and 303 are also useful when the orthosis 210 is arranged with a lateral tilt or for a patient with torticollis or other tilt. For example, the health worker can arrange the bands 302 and 303 differently on the left arm 213 and the right arm 214 such that the bands 302 and 303 create a lateral bias on the upper collar 11′ in the direction of the left or right arm 213 and 214. This may help counteract a head tilt. As another example, the health worker lowers one arm 214, raises the other arm 213, and then instructs the patient to perform exercises against the bands 302 and 303, such as by lifting the patient's head away from the lowered arm 213. This tilts the upper collar 11′ to level, lifting the strut 224 of the lowered arm 213. The health worker can arrange resistive or assistive force for this exercise. By placing the bands 303 on the lowered arm 213, and/or by placing the bands 302 on the raised arm 214, the health worker can create assistive force. Conversely, by placing the bands 302 on the lowered arm 213, and/or by placing the bands 303 on the raised arm 214, the health worker can create resistive force.

Vertical movement of either or both of the arms 213 and 214 is possible when the top and bottom pegs 286 and 287 enable it. Turning to the section views of FIGS. 12A and 12B, which is taken along the line 12-12 in FIG. 11B, the top and bottom pegs 286 and 287 are inserted into the top-most and bottom-most sockets 285. This limits vertical movement of the strut 224 with respect to the post 220. The bottom 291 of the slot 288 quickly contacts the bottom peg 287, thereby preventing vertical movement of the strut 224 with respect to the post 220. In FIG. 12B, the top and bottom pegs 286 and 287 are removed, and the strut 224 can move upward on the post 220 without limitation. The long band 302 stretches considerably.

The orthoses 10 and 210 are custom-manufactured and fit for a patient. To build this, the health worker creates a nearly exact replica of the patient's anatomy. To do this, the health worker creates a positive mold 310. FIG. 13 is a top perspective view of a positive mold 310 created from a scan of a patient's neck area. The positive mold 310 is printed with a 3D printing additive manufacturing process. The positive mold 310 replaces a conventional foam mold and is used as a foundation for constructing the cervical collar orthosis 210 shown in the other drawings.

To create the positive mold 310, a health worker scans the patient around his neck, including his upper chest and the lower portion of his head. Several types of scanners are suitable, one such including a true depth scanner, which projects thousands of infrared dots onto an object to create a detailed three-dimensional map of the object. This map is then read into a 3D printer. The 3D printer prints a shell 311 that has a thin sidewall 312 with opposed inner and outer surfaces 313 and 314 separated by a slight thickness 315. That thickness 315 is preferably constant over the entire extent of the shell 311.

The shell 311 shown in FIG. 13 is constructed from four separate panels joined along seams 316. The seams 316 are held together by joinder assemblies 317. In the embodiment shown in FIG. 13, the joinder assemblies 317 include a ridge 318 spaced inboard of the seam 316 slightly on one of the panels and a plurality of fingers 319 extending beyond the seam 316 from the adjacent panel. The fingers 319 fit and snap over the ridge 318 to bring the two adjacent panels together tightly.

Once built, the shell 311 represents a nearly exact replica of the patient's anatomy. From this, a manufacturer can construct an orthosis such as the exemplary orthoses 10 and 210 described above. The positive mold 310 and the orthoses 10 and 210 shown and described in this specification are exemplary of many types of orthoses. This specification describes in detail a process for creating the positive mold 310 and the cervical collar orthoses 10 and 210 but it is not so limited; the process can be used to construct other orthoses such as knee braces, arm splints, hip orthotics, and other medical devices. Indeed, FIGS. 14-23 illustrate scans of other body parts or sections. The description should be understood to apply to methods for scanning and forming molds based on these and other body parts or sections, without limitation.

A preferred embodiment is fully and clearly described above so as to enable one having skill in the art to understand, make, and use the same. Those skilled in the art will recognize that modifications may be made to the description above without departing from the spirit of the specification, and that some embodiments include only those elements and features described, or a subset thereof. To the extent that modifications do not depart from the spirit of the specification, they are intended to be included within the scope thereof.

What is claimed is:

1. An adjustable orthosis comprising:
a lower collar configured to rest on a patient torso;
an upper collar configured to support a patient head;
an arm extending between the lower collar and the upper collar, the arm supporting the upper collar at an elevated position with respect to the lower collar; and
the arm enables rotational movement of the upper collar with respect to the lower collar through a rotational range of movement in a transverse plane.

2. The adjustable orthosis of claim 1, further comprising:
a track formed in the lower collar, the track having opposed first and second ends;
wherein the arm is carried in the track for movement through the rotational range of movement between the first and second ends of the track.

3. The adjustable orthosis of claim 2, further comprising first and second stops applicable to the track such that, when the first and second stops are applied to the track between the first and second ends, the first and second stops limit the rotational range of movement to between the first and second stops.

4. The adjustable orthosis of claim 1, wherein the arm further enables pitching movement of the upper collar with respect to the lower collar through a range of movement in a sagittal plane.

5. The adjustable orthosis of claim 4, wherein the arm comprises:
a post coupled to the lower collar;
a strut coupled to the upper collar;
a spindle defining an axis of rotation for the pitching movement, wherein the spindle is fixed with respect to the strut;
an inner plate mounted for rotation on the spindle;
an outer plate mounted for rotation on the spindle; and
the post is mounted for rotation on the spindle with respect to the strut.

6. The adjustable orthosis of claim 5, wherein:
the inner plate has a first set of teeth and a block outboard of the first set of teeth;
the outer plate has a second set of teeth engaged with the first set of teeth, and a second block outboard of the second set of teeth; and
the post has a third set of teeth engaged with the second set of teeth.

7. The adjustable orthosis of claim 6, wherein:
the spindle is carried on a base plate, and the base plate includes a third block; and
during pitching movement, the base plate rotates on the strut with respect to the post, and the third block moves between the first and second blocks, wherein the first and second blocks confront the third block to limit rotational movement of the base plate and the strut with respect to the post.

8. The adjustable orthosis of claim 7, wherein the arm enables vertical movement of the upper collar with respect to the lower collar through a vertical range of movement in a frontal plane.

9. The adjustable orthosis of claim 8, wherein:
the strut includes a channel;
the base plate includes a rail fit into the channel;
wherein, during vertical movement of the upper collar with respect to the lower collar, the rail of the base plate slides within the channel of the strut; and
the post is coupled to the base plate by the inner and outer plates, such that movement of the base plate with respect to the strut imparts movement of the post with respect to the strut.

10. The adjustable orthosis of claim 8, further comprising stops carried by the base plate which confront the strut to limit vertical movement of the upper collar with respect to the lower collar to within a defined range.

11. An adjustable orthosis comprising:
a lower collar configured to rest on a patient torso;
an upper collar configured to support a patient head;
an arm extending between the lower collar and the upper collar, the arm supporting the upper collar at an elevated position with respect to the lower collar; and
the arm enables pitching movement of the upper collar with respect to the lower collar through a pitching range of movement in a sagittal plane.

12. The adjustable orthosis of claim 11, wherein the arm comprises:
a post coupled to the lower collar;
a strut coupled to the upper collar;
a spindle carried on the strut, the spindle defining an axis of rotation for the pitching movement; and
the post is mounted for rotation on the spindle with respect to the strut.

13. The adjustable orthosis of claim 12, further comprising:
inner and outer plates between the post and the strut;
an inner plate has a first set of teeth and a block outboard of the first set of teeth;
the outer plate has a second set of teeth engaged with the first set of teeth, and a second block outboard of the second set of teeth; and
the post has a third set of teeth engaged with the second set of teeth.

14. The adjustable orthosis of claim 13, further comprising a base plate including a third block, wherein, during pitching movement, the base plate rotates on the strut with respect to the post, and the third block moves between the first and second blocks, wherein the first and second blocks confront the third block to limit rotational movement of the base plate and the strut with respect to the post.

15. The adjustable orthosis of claim 12, further comprising bands extending between the post and the strut, the bands imparting a bias therebetween.

16. The adjustable orthosis of claim 11, further comprising:
a pitch assembly in the arm; and
first and second stops applicable to the arm such that, when the stops are applied to the pitch assembly, the stops limit the pitching range of movement to between the first and second stops.

17. The adjustable orthosis of claim 12, wherein the arm enables vertical movement of the upper collar with respect to the lower collar through a vertical range of movement in a frontal plane.

18. The adjustable orthosis of claim 14, wherein:
the strut includes a channel;
the base plate includes a rail fit into the channel;
wherein, during vertical movement of the upper collar with respect to the lower collar, the rail of the base plate slides within the channel of the strut; and
the post is coupled to the base plate by the inner and outer plates, such that movement of the base plate with respect to the strut imparts movement of the post with respect to the strut.

19. An adjustable orthosis comprising:
a lower collar configured to rest on a patient torso;
an upper collar configured to support a patient head; and
an arm extending between the lower and upper collars, the arm supporting the upper collar at an elevated position with respect to the lower collar;
wherein the arm supports the upper collar at an elevated position with respect to the lower collar and enables a range of movement of the upper collar with respect to the lower collar in at least one of three planes of movement.

20. The adjustable orthosis of claim 19, wherein the arm includes stops to limit the range of movement of the upper collar within a defined range.
